(19) United States
(12) Patent Application Publication
(10) Pub. No.: US 2026/0204361 A1
(43) Pub. Date: Jul. 16, 2026

(54) DE NOVO DRUG DESIGN USING REINFORCEMENT LEARNING

(71) Applicant: Recursion Pharmaceuticals, Inc., Salt Lake City, UT (US)

(72) Inventors: Marco CAROBENE, Dundee (GB); Aleksandra Dominika KALISZ, Dundee (GB); Marius URBONAS, Dundee (GB)

(21) Appl. No.: 19/135,338

(22) PCT Filed: Dec. 15, 2023
(86) PCT No.: PCT/GB2023/053273

§ 371 Date: Jun. 3, 2025

(30) Foreign Application Priority Data
Dec. 16, 2022 (GB) 2219100.1

(51) Int. Cl. G16C 20/50; G06N 3/092; G16C 20/30; G16C 20/70

(52) U.S. Cl. CPC G16C 20/50; G06N 3/092; G16C 20/30; G16C 20/70

(57) ABSTRACT

The invention relates to methods for the computational design of compounds, such as drugs. The invention relates to methods for improved computational models through reinforcement learning, to be used in the de novo design of drugs that have certain desired properties. The invention involves training a generative model to generate compounds or molecules from scratch, scoring said generated compounds based on their properties, e.g. biological properties, relative to a desired property profile, e.g. of a candidate compound, and then using a reinforcement learning algorithm to update a current policy of the generative model. The method involves repeating the compound generation and current policy update steps in an iterative manner. The invention involves selecting high scoring compounds, and training a Bayesian model for approximating a score and uncertainty of compounds as a function of structural features of said selected compounds, which is used to improve quality and diversity of generated compounds.

### FIELD OF THE INVENTION

The invention relates to methods and systems for the computational design of compounds, such as drugs. In particular, the invention relates to methods for improved computational models through reinforcement learning, to be used in the de novo design of drugs that have certain desired properties, and to the drugs designed using these methods and systems.

### BACKGROUND

Drug discovery is the process of identifying candidate compounds for progression to the next stage of drug development, e.g. pre-clinical trials. Such candidate compounds are required to satisfy certain criteria for further development. Modern drug discovery involves the identification and optimisation of initial screening ‘hit’ compounds (‘lead identification’). In particular, identified initial screening hit compounds may be compounds that exhibit at least some properties that are desired according to a drug discovery project under consideration, and then these initial hits are optimised to obtain compounds that have more of the specified desired properties. Specifically, initial hit compounds need to be optimised relative to required criteria, which can include the optimisation of a number of different properties. The properties to be optimised can include, for instance: efficacy/potency against a desired target; selectivity against non-desired targets; low probability of toxicity; and, good drug metabolism and pharmacokinetic properties (ADME). Only compounds satisfying the specified requirements become candidate compounds that can continue to the drug development process.

The identification of initial screening hit compounds may be performed in different ways. For instance, high-throughput screening is a technique for conducting a relatively large number of automated biological or chemical tests in a relatively short period of time to identify relevant compounds, e.g. compounds that are active against a defined biological target. This allows large compound libraries to be screened relatively quickly, but is limited to compounds included in such libraries.

Another technique for identifying initial screening hit compounds is virtual screening. This is a computational technique to search libraries of compounds to identify those structures which are most likely to exhibit desired properties. This technique may be useful when the virtual compounds are readily available or easy to synthesise. However, only a small fraction of drug-like chemical space may be searched according to such techniques, even with up-to-date computational resources.

A computational technique for identifying and optimising hit compounds is de novo design. This is a technique for exploring chemical space using search or optimisation procedures to explicitly generate compounds. This can involve the incremental construction of compounds from scratch, for instance by sequentially selecting and adding fragments to be included in the designed compound. In de novo design, fewer compounds are generally considered compared with virtual screening. However, de novo design algorithms may in principle explore the full chemical space, but focus on particular areas relevant to the particular desired properties of a certain drug discovery project.

A de novo drug design project needs to consider how best to generate or assemble compounds, how to evaluate generated compounds relative to a desired compound profile, and how to effectively search chemical space to generate better compounds relative to how they are being evaluated.

Different approaches for generating compounds for this purpose are possible. For instance, one way may be via the use of a recurrent neural network (RNN) model. An RNN is a useful tool for the sequential generation of objects, such as compounds. Such generative models learn a probability distribution over a large training set of compounds to relate molecular structures to molecular properties. The trained model can then be executed to obtain compounds similar to those from the training set and which are predicted to exhibit one or more desired properties.

Different techniques for improving generative models may also be used. For instance, reinforcement learning (RL) may be used to increase the likelihood of compounds that are created using a generative model exhibit more desired properties, and/or are compounds that may readily be synthesised. In particular, RL may be used to improve the actions taken by a model to choose which chemical fragments to include in a compound, for instance. This is achieved by requiring the model to maximise a reward associated with its choices, the reward being indicative of whether certain objectives are being achieved, e.g. the compound exhibits desired drug-like properties. Expressed differently, RL can be used to learn an optimal (or near optimal) policy that the model follows in order to maximise rewards, i.e. in this case the likelihood that the generated compounds are optimised relative to a target or desired property profile.

There continues to be a need for improved methods to generate compounds as part of a de novo design process, in particular to generate ‘better’ compounds that are more likely to be optimised relative to a target profile of a lead compound or clinical candidate. By generating more optimal compounds, a more efficient process to obtain candidate compounds becomes more likely. This is important as increased efficiency leads to cost and time savings associated with a drug discovery project.

In more detail, the drug discovery process can involve making/synthesising a significant number of compounds during the optimisation from lead identification to clinical candidate compounds. In particular, those compounds which are synthesised are measured to determine their properties, such as biological activity. However, the number of compounds that it may be desired or required to make as part of a particular drug discovery project will far outnumber—likely by orders of magnitude—the number of compounds that can be synthesised and tested. The results of the measurements of synthesised compounds are therefore analysed and used to inform a decision on which compounds to synthesise next to maximise the likelihood of obtaining compounds with further improved properties relative to the various criteria required by a candidate compound.

The synthesis and subsequent measurement of biological activity of one or more compounds at a particular stage is referred to as a design cycle (or iteration) of the drug discovery process. Typically, a set of compounds is synthesised and tested at each design cycle of the process as this is more efficient than synthesising and testing a single compound at a time. However, a level of available resources usually means that there is an upper limit on the number of compounds in a set that can be synthesised at any given design cycle.

During a drug discovery project, many hundreds or even thousands of compounds typically are synthesised across several design cycles before a candidate compound is found. This is a lengthy, expensive, and inefficient process: synthesis of a single compound can cost thousands of pounds, and it can take three to five years on average to obtain a single candidate compound. Improved computational techniques for designing and testing compounds—i.e. de novo design techniques—that are closer to the target profile of a clinical candidate are therefore crucial for reducing the number of required design cycles, i.e. the number of compounds that need to be synthesised and tested, and so are crucial for increasing the efficiency of a drug optimisation process.

It is against this background to which the present invention is set.

### SUMMARY OF THE INVENTION

According to an aspect of the present invention there is provided a method for de novo drug design. The method comprises computer-implemented steps of: receiving a training set of a plurality of training compounds, each training compound having a plurality of structural features; defining a desired property profile indicative of one or more desired properties of a drug compound to be designed; training a generative model using the training set to obtain a prior probability distribution of the structural features in the training set; generating an initial set of one or more compounds using the generative model, wherein generating each compound comprises sampling the prior probability distribution; determining a compound score for each compound in the initial set, the compound score being indicative of a proximity of a property profile of the respective compound to the desired property profile; and determining a current policy of the generative model, the current policy being determined by modifying the prior probability distribution based on the determined compound scores using a reinforcement learning algorithm.

The method comprises iteratively performing computer-implemented steps of: generating a set of a plurality of compounds using the generative model, wherein generating each compound comprises sampling the current policy of the generative model; determining the compound score for each compound in the set; storing the set of generated compounds along with the associated determined compound scores; and updating the current policy of the generative model by modifying the probability distribution based on the determined compound scores of the plurality of compounds in the set using the reinforcement learning algorithm. The iterative steps are performed until a stop condition is satisfied.

The method comprises performing computer-implemented steps of: selecting a plurality of compounds from the stored sets of generated compounds, the selected compounds being the generated compounds having the highest compound scores; and training, using the selected plurality of compounds, a Bayesian model for approximating the compound score of compounds as a function of structural features of said compounds.

The method comprises computer-implemented steps of: generating a further set of one or more compounds by sampling the current policy of the generative model; executing the trained Bayesian model for each generated compound in the further set to obtain an uncertainty value associated with a predicted output of the Bayesian model, the uncertainty value being indicative of dissimilarity of the respective compound relative to the selected plurality of compounds; and determining a further compound score for each generated compound in the further set based on a combination of the compound score and the uncertainty value for the respective compound.

The method may comprise a computer-implemented step of selecting one or more of the compounds in the further set having the highest further compound scores.

In some examples, selecting the one or more compounds comprises: selecting each of the compounds having a further compound score greater than a defined further compound score threshold value; or, selecting a prescribed number of the compounds, wherein the selected compounds are those in the further set with the highest further compound scores.

The method may comprise synthesising the one or more of the selected compounds to determine one or more biological properties of said compounds. That is, each selected compound may be synthesised and tested experimentally, e.g. via in vitro synthesis/testing, in order to ascertain certain biological properties of the respective compound.

The method may comprise computer-implemented steps of: adding the synthesised compounds to the training set to obtain an updated training set; retraining the generative model using the updated training set to obtain an updated prior probability distribution of the structural features in the updated training set; generating an updated initial set of one or more compounds using the updated generative model, wherein generating each compound comprises sampling the updated prior probability distribution; determining the compound score for each compound in the updated initial set; and, determining a current policy of the updated generative model, the current policy being determined by modifying the updated prior probability distribution based on the determined compound scores using the reinforcement learning algorithm.

The method may comprise, prior to adding the synthesised compounds to the training set, computer-implemented steps of: determining an actual compound score for each of the synthesised compounds, the actual compound score being indicative of a proximity of an actual property profile, based on the determined one or more biological properties, of the respective synthesised compound to the desired property profile; and, selecting a subset of the synthesised compounds, the subset including the synthesised compounds having the highest actual compound scores. Adding the synthesised compounds to the training set may comprise adding only those compounds in the selected subset to the training set.

The method may comprise iteratively performing computer-implemented steps of: generating a set of a plurality of compounds using the updated generative model, wherein generating each compound comprises sampling the current policy of the updated generative model; determining the compound score for each compound in the set; storing the set of generated compounds along with the associated determined compound scores; and, updating the current policy of the updated generative model by modifying the probability distribution based on the determined compound scores of the plurality of compounds in the set using the reinforcement learning algorithm. The iterative steps may be performed until the stop condition is satisfied.

The method may comprise performing computer-implemented steps of: selecting a plurality of compounds from the stored sets of generated compounds, the selected compounds being the generated compounds having the highest compound scores; and, retraining the Bayesian model using the selected plurality of compounds.

The method may comprise computer-implemented steps of: generating a second further set of one or more compounds by sampling the current policy of the updated generative model; executing the retrained Bayesian model for each generated compound in the second further set to obtain the uncertainty value associated with the predicted output of the Bayesian model; and, determining the further compound score for each generated compound in the second further set based on a combination of the compound score and the uncertainty value for the respective compound.

The method may comprise a computer-implemented step of selecting one or more of the compounds in the second further set having the highest further compound scores. The method may comprise synthesising one or more of the selected compounds to determine one or more biological properties of said compounds.

The method may comprise computer-implemented steps of: adding the selected compounds to the training set to obtain an updated training set; retraining the generative model using the updated training set to obtain an updated prior probability distribution of the structural features in the updated training set; generating an updated initial set of one or more compounds using the updated generative model, wherein generating each compound comprises sampling the updated prior probability distribution; determining the compound score for each compound in the updated initial set; and, determining a current policy of the updated generative model, the current policy being determined by modifying the updated prior probability distribution based on the determined compound scores using the reinforcement learning algorithm.

The method may comprise iteratively performing computer-implemented steps of: generating a set of a plurality of compounds using the updated generative model, wherein generating each compound comprises sampling the current policy of the updated generative model; determining the compound score for each compound in the set; storing the set of generated compounds along with the associated determined compound scores; and, updating the current policy of the updated generative model by modifying the probability distribution based on the determined compound scores of the plurality of compounds in the set using the reinforcement learning algorithm. The iterative steps may be performed until the stop condition is satisfied.

The method may comprise performing computer-implemented steps of: selecting a plurality of compounds from the stored sets of generated compounds, the selected compounds being the generated compounds having the highest compound scores; and, retraining the Bayesian model using the selected plurality of compounds.

The method may comprise computer-implemented steps of: generating a second further set of one or more compounds by sampling the current policy of the updated generative model; executing the retrained Bayesian model for each generated compound in the second further set to obtain the uncertainty value associated with the predicted output of the Bayesian model; and, determining the further compound score for each generated compound in the second further set based on a combination of the compound score and the uncertainty value for the respective compound.

The method may comprise a computer-implemented step of selecting one or more of the compounds in the second further set having the highest further compound scores.

The method may comprise a computer-implemented step adding the selected compounds from the second further set to the updated training set to obtain a second updated training set.

The method may comprise synthesising one or more of the selected compounds from the second further set to determine one or more biological properties of said compounds.

In some examples, for each of the selected compounds, the respective selected compound may be synthesised only if it satisfies a prescribed condition. Optionally, the prescribed condition may be that the further compound score of the respective selected compound is greater than a prescribed threshold further compound score.

The method may comprise a computer-implemented step adding the synthesised compounds to the updated training set.

In some examples, one or more biological properties may be known for each training compound in the training set.

The compound score of each generated compound in the initial set and the set may be determined using a defined reward function.

The compound score of each generated compound in the further set may be determined using the defined reward function.

The defined reward function may be determined based on predicted biological activity of the respective compound against one or more defined targets.

In some examples, predicted biological activity may be determined using a trained machine learning model configured to receive one or more structural features as input and to output predicted biological activity of the respective compound as a function of the one or more structural features of the respective compound.

The defined reward function may be determined according to

$$\frac{1}{2.5} ( {\hat{x}}_{1} + {\hat{x}}_{2} + 0.5 * {x}_{Q E D} )$$

where \hat{x}₁ϵ[0,1] is a probability of half-maximal inhibitory concentration being greater than a defined value against a first defined target, \hat{x}₂ϵ[0,1] is a probability of half-maximal inhibitory concentration being greater than the defined value against a second defined target, and x_{QED}ϵ[0,1] is a defined metric indicative of quantitative estimate of drug-likeness.

The defined reward signal may be determined according to

$${R}_{e x t} = \frac{1}{8.5} ( 3 {f}_{1} ( {\hat{x}}_{1} ) + 2 {f}_{2} ( {\hat{x}}_{2} ) + {f}_{3} ( {x}_{MW} ) + {f}_{4} ( {x}_{A R C} ) + 1.5 * {x}_{Q E D} )$$

where \hat{x}₁ϵ [symbol] is predicted potency against a defined target, \hat{x}₂ϵ [symbol] is a predicted acid dissociation constant, x_{MW}ϵ [symbol] is calculated molecular weight, x_{ARC}Σ [symbol] is calculated aromatic ring count, x_{QED}ϵ[0,1] is a metric indicative of quantitative estimate of drug-likeness, and

$${\{ {f}_{i} : ℝ \to [ 0 , 1 ] \}}_{i = 1}^{4}$$

are defined nonlinear desirability transformations to specify defined ranges for each respective property \hat{x}₁, \hat{x}₂, x_{MW}, x_{ARC}.

In some examples, determining the compound score comprises: applying a plurality of property scorer functions to the respective compound to obtain a plurality of property scores for the compound, each property score being indicative of a proximity of a value of one of the properties of the compound to a desired value of the respective property in the desired property profile; and applying a defined reward function to the plurality of obtained property scores to obtain the compound score for the respective compound.

The compound score of each generated compound in the further set may be determined as the predicted output of the executed Bayesian model.

The method may comprise determining an intrinsic compound score for each generated compound based on the respective uncertainty value thereof.

In some examples, determining the intrinsic compound score comprising determining a normalised value of the uncertainty value. The normalised value may be determined to be within a defined normalised range, e.g. [−1, 1].

The normalised value may be determined based on a median value and a maximum value of uncertainty values of a plurality of compounds generated using the prior probability distribution.

The intrinsic compound score \hat{r}(m) may be determined according to

$$\hat{r} ( m ) = β * min \{ max \{ \frac{{σ}_{G P} ( m ) - M}{S} , - 1 \} , 1 \}$$

where is σ_{GP}(m) the uncertainty value of the respective generated compound, M is the median value of the uncertainty values of the plurality of compounds generated using the prior probability distribution, S is the maximum value of the uncertainty values of the plurality of compounds generated using the prior probability distribution, and β is a scaled value of a mean of the compound score of the generated compounds having the highest compound scores.

The further compound score may be determined as a linear combination of the compound score and the intrinsic compound score associated with the respective compound.

The linear combination may be a linear combination of weighted values of the compound score and the intrinsic compound score.

A weighting of the intrinsic compound score may be increased relative to a weighting value of the compound score in the linear combination in subsequent iterations of the method.

The stop condition may include a prescribed number of iterations have been performed.

The stop condition may include that the compound score of one or more compounds in the set satisfies a threshold condition. Optionally, the threshold condition may be that the compound score of one or more compounds in the set is greater than a defined threshold compound score.

The stop condition may include that a rate of change of the compound scores across iterations falls below a prescribed level.

The Bayesian model may be a Gaussian Process model.

The generative model may be a recurrent neural network model.

The training compounds and generated compounds may be represented as SMILES strings.

The generated compounds may be represented as CHUCKLES strings.

The training set may comprise: a first training set of a first plurality of training compounds; and, a second training set of a second plurality of training compounds, the second training set being different from the first training set. Training the generative model may comprise: using the first training set to obtain a first prior probability distribution of the structural features in the first training set; and, using the second training set to modify the first prior probability distribution to obtain the prior probability distribution, wherein the prior probability distribution incorporates a distribution of the structural features in the second training set.

The compound score may be a one-dimensional value.

Using the reinforcement learning algorithm may comprise applying a policy gradient method to minimise a defined loss function.

Applying the policy gradient method may comprise applying a proximal policy optimisation method, which may comprise including one or more terms in the loss function defining clipped probability ratios.

Applying the policy gradient method may comprise including a Kullback-Leibler divergence term in the loss function.

Applying the policy gradient method may comprise including a likelihood regularisation term in the loss function.

Applying the policy gradient method may comprise including a maximum mean discrepancy term in the loss function.

Applying the maximum mean discrepancy method may comprise defining a metric to measure a difference between two trajectory distributions indicative of respective compounds. The compounds may be represented as extended connectivity fingerprints (ECFP). The metric may be defined to determine a kernel of similarity between the compounds.

The kernel of similarity may be defined to be a Tanimoto similarity.

Applying the policy gradient method may comprise including each of: a Kullback-Leibler divergence term; a likelihood regularisation term; and, a maximum mean discrepancy term, in the loss function.

Applying the policy gradient method may comprise applying a generalised advantage estimation scheme.

The second training set may comprise fewer compounds than the first training set.

The second training set may be composed of compounds that exhibit at least some desired properties in accordance with the desired property profile. Optionally, the second training set may be composed of compounds of a defined type. Optionally, the defined type may be a kinase.

The first training set may be composed of compounds from a defined region of compound space. The second training set may be composed of compounds from a sub-region of the defined region of compound space.

The plurality of compounds in the received training set may each be represented as SMILES strings. The method may comprise converting the SMILES strings to represent the plurality of compounds as CHUCKLES strings. The generative model may be trained using the plurality of compounds represented as CHUCKLES strings.

The generated compounds may be represented as SELFIES strings.

The plurality of compounds in the received training set may each be represented as SMILES strings. The method may comprise converting the SMILES strings to represent the plurality of compounds as SELFIES strings. The generative model may be trained using the plurality of compounds represented as SELFIES strings.

Generating each compound may comprise sequentially sampling the current policy to obtain a string of tokens representing the generated compound.

The current policy may be sampled until a token indicating that the generation process is complete is sampled.

Generating each compound may comprise:
obtaining a partial string of tokens representing a partial compound;
evaluating the partial compound relative to the desired property profile;
updating the current policy based on the evaluation of the partial compound; and,
sequentially sampling the updated current policy starting from the partial string of tokens.

A final token of the partial string may be a dot, indicating that the partial compound is a valid compound in CHUCKLES notation.

A subset of tokens in the string of tokens may be predetermined, indicating that part of the generated compound is predetermined.

The part of the generated compound corresponding to its core may be predetermined.

The current policy may comprise a core policy and an agent policy. Generating each compound may comprise sampling the core policy at locations of the generated string corresponding to the core of the compound and sampling the agent policy at locations of the string corresponding to non-core parts of the compound.

The core policy may be fixed. Updating the current policy may comprise updating only the agent policy.

The method may comprise tracking a policy mask during the compound generation step. The policy mask may be indicative at each sampling instance of whether the core policy or agent policy is sampled to obtain a given token in the string of tokens representing the generated compound. The current policy may be updated based on tokens sampled in accordance with the agent policy.

The one or more desired properties may include one or more of a desired: activity; selectivity; toxicity; absorption; distribution; metabolism; and, excretion.

The method may comprise analysing the properties of the synthesised compounds relative to the desired property profile. The method may comprise adding only those synthesised compounds to the second training set that are within a defined proximity of the desired property profile.

The one or more selected compounds may be a candidate drug or therapeutic molecule having a desired biological, biochemical, physiological and/or pharmacological activity against a predetermined target molecule.

The predetermined target molecule may be an in vitro and/or in vivo therapeutic, diagnostic or experimental assay target.

The candidate drug or therapeutic molecule may be for use in medicine; for example, in a method for the treatment of an animal, such as a human or non-human animal.

According to an aspect of the present invention there is provided a method for de novo drug design. The method comprises computer-implemented steps of:
receiving a trained generative model that includes a prior probability distribution of structural features of a training set of a plurality of training compounds each having a plurality of structural features;
generating an initial set of one or more compounds using the generative model, wherein generating each compound comprises sampling the prior probability distribution;
determining, using a defined reward function, a compound score for each compound in the initial set, the compound score being indicative of a proximity of a property profile of the generated initial compound relative to the desired property profile;
determining a current policy of the generative model, the current policy being determined by modifying the prior probability distribution based on the determined score using a reinforcement learning algorithm; and, generating a new set of one or more compounds using the current policy of the generative model.

The method may comprise: receiving the training set of the plurality of training compounds; and, training the generative model using the training set to obtain the prior probability distribution.

Training the generative model using the training set may comprise modifying a predefined probability distribution based on the training set to obtain the prior probability distribution.

Determining the current policy may comprise applying a proximal policy optimisation method to minimise a defined loss function that comprises one or more terms defining clipped probability ratio. Applying the proximal policy optimisation method may comprise including at least one of the following in the loss function: a Kullback-Leibler divergence term; a likelihood regularisation term; and, a maximum mean discrepancy term. Optionally, the loss function includes each of the Kullback-Leibler divergence term, the likelihood regularisation term, and the maximum mean discrepancy term.

The generated compounds may be represented as SELFIES strings.

Generating each compound may comprise sequentially sampling the current policy to obtain a string of tokens representing the respective compound. A subset of tokens in the string of tokens may be predetermined, indicating that part of the generated compound is predetermined.

According to an aspect of the invention there is provided a computer-implemented method for de novo drug design. The method comprises iteratively performing steps of:
generating a set of a plurality of compounds using a defined generative model, wherein generating each compound comprises sampling a current policy of the generative model, the current policy including a probability distribution of compound structural features;
determining, using a defined reward function, a compound score for each compound in the set, the compound score being indicative of a proximity of a property profile of the respective compound to a defined property profile indicative of one or more desired properties of a drug compound to be designed;
storing the set of generated compounds along with the associated determined compound scores; and,
updating the current policy of the generative model by modifying the probability distribution based on the determined compound scores using a reinforcement learning algorithm, for a predefined number of iterations.

The method may comprise:
selecting a plurality of compounds from the stored sets of generated compounds, the selected compounds being the generated compounds having the highest compound scores; and,
training, using the selected plurality of compounds, a Gaussian Process model for approximating the compound score of compounds as a function of structural features of said compounds.

The method may comprise:
generating a further set of one or more compounds by sampling the current policy of the generative model;
executing the trained Gaussian Process model for each generated compound in the further set to obtain an uncertainty value associated with a predicted output of the Gaussian Process model, the uncertainty value being indicative of dissimilarity of the respective compound relative to the selected plurality of compounds; and,
determining a further compound score for each generated compound in the further set based on a combination of the compound score and the uncertainty value for the respective compound.

The compound score of each generated compound in the further set may be determined using the defined reward function or obtained from the executed Gaussian Process model.

The method may comprise: updating the current policy of the generative model by modifying the probability distribution based on the determined further compound scores using the reinforcement learning algorithm; generating a set of one or more compounds by sampling the current policy of the generative model; determining the further compound score for each generated compound in the further set; and, storing the further set of generated compounds along with the associated determined further compound scores.

The method may comprise:
selecting a further plurality of compounds from the stored sets and further sets of generated compounds, the selected compounds being the generated compounds having the highest compound scores or further compound scores; and,
re-training, using the selected further plurality of compounds, the Gaussian Process model for approximating the compound score of compounds as a function of structural features of said compounds.

According to another aspect of the invention there is provided a compound identified by the method defined above.

According to another aspect of the present invention there is provided a non-transitory, computer-readable storage medium storing instructions thereon that when executed by a computer processor causes the computer processor to perform the method defined above.

According to another aspect of the present invention there is provided a computing device for computational drug design. The computing device comprises an input arranged to receive a trained generative model including a prior probability distribution of structural features of a training set of a plurality of training compounds. The computing device comprises a processor arranged to: generate an initial compound in accordance with the prior probability distribution using the trained generative model; define a desired property profile indicative of one or more desired properties of a drug to be designed; obtain a score associated with the generated initial compound, the score being indicative of a property profile of the generated initial compound relative to the desired property profile; determine a current policy of the generative model, the current policy being determined by modifying the prior probability distribution based on the determined score using a reinforcement learning algorithm; and, generate a new compound in accordance with the current policy. The computing device comprises an output arranged to output the generated new compound.

The processor may be configured to perform a method as defined above.

### BRIEF DESCRIPTION OF THE DRAWINGS

Examples of the invention will now be described with reference to the accompanying drawings, in which:

FIG. 1 shows an overview of a pipeline or architecture of a system for the de novo design of compounds in accordance with an example of the invention;

FIG. 2 schematically illustrates how a trained generative model of the system of FIG. 1 is used to generate a new sequence corresponding to a new compound;

FIG. 3 schematically illustrates updating the generative model of FIG. 2 to optimise a reward signal associated with compounds generated by the model;

FIG. 4 schematically illustrates the structure of the core of an example molecule for which different attachments are to be generated by the system of FIG. 1 in accordance with an example of the invention;

FIG. 5 shows the steps of a method performed by the system of FIG. 1 in accordance with an example of the invention;

FIG. 6 illustrates an example of the performance of different reinforcement learning (RL) methods performed by an RL agent of the system of FIG. 1, where FIG. 6(a) shows mean reward signal of the top 10% of generated molecules against number of iterations, and FIG. 6(b) shows the entropy of generated molecules against number of iterations;

FIGS. 7(a) and 7(b) show mean reward signal of the top 10% of generated molecules and entropy, respectively, against number of iterations in an illustrative example of the performance of different methods from FIG. 6;

FIGS. 8(a) and 8(b) show mean reward signal of the top 10% of generated molecules and entropy, respectively, against number of iterations in an illustrative example of the performance of further different methods from FIG. 6; and,

FIG. 9 illustrates an example of the performance of a method performed by an RL agent of the system of FIG. 1, where FIG. 9(a) shows mean reward signal of the top-scoring generated molecules against number of iterations, FIG. 9(b) shows the similarity of top-scoring generated molecules against number of iterations, and FIG. 9(c) shows number of unique scaffolds of top-scoring generated molecules against number of iterations.

### DETAILED DESCRIPTION

Molecular or drug design can be considered a multi-dimensional optimisation problem that uses the hypothesis generation and experimentation cycle to advance knowledge. Each compound design can be considered a hypothesis which is falsified in experimentation. The experimental results are represented as structure-activity relationships, which construct a landscape of hypotheses as to which chemical structure is likely to contain the desired characteristics. The process of drug design is also an optimisation problem as each project starts out with a product profile—i.e. target function—of desired, specified attributes. However, even though the objective can be accurately described, it has previously been an expensive and difficult challenge to find an optimal solution. One particular difficulty with this type of problem is to effectively construct the landscape of hypotheses across the vast space of feasible solutions from a relatively limited knowledge base of experimental results.

The drug discovery process is typically performed in iterations or cycles that involve steps of designing, making and testing compounds. At each iteration, a set of molecules or compounds is synthesised, and their biological properties are measured. The activities are analysed, and a new set of compounds is proposed, based on what has been learned from previous iterations. This process is repeated until a clinical candidate is found. As well as activity, the measured biological properties can include one or more of selectivity, toxicity, affinity, absorption, distribution, metabolism, and excretion.

Many compounds typically need to be synthesised across several design cycles before a candidate compound is found. Given that synthesis of even a single compound can be expensive, and that it can typically take several years to obtain a single clinical candidate compound, then it is apparent that the drug discovery process is generally expensive and time consuming.

De novo design techniques can be used to increase the efficiency of the drug discovery process by utilising computational methods to assist with, or fully perform, one or more of the steps of the design cycle. For instance, computational models—e.g. machine learning models—may be built and trained for use at the testing step to relate structural features of compounds to biological activity. Such models can predict the biological activity of a compound based on its molecular structure, and these predictions may be used to optimise a selection of which compounds to synthesise with the aim of obtaining a clinical candidate.

At the design step of an iteration or cycle, computational models may be used to generate compounds from scratch in relevant areas of chemical space to a target property profile associated with a particular drug discovery project. In particular, generative models may be used to learn a distribution of real drug-like compounds from training data, and then generate new structures that may exhibit desired properties based on this learned distribution.

Reinforcement learning is a technique that may then be used to improve the trained generative models by further tuning the models to make better selections when creating compounds. That is, reinforcement learning can be used to modify the previously trained models to generate compounds that are more likely to exhibit more desired properties according to a target profile.

The present invention is advantageous in that it provides improved methods and systems for de novo drug design. The invention applies reinforcement learning techniques to generative models in order to design molecules or compounds as part of a drug discovery project. In particular, the present invention is advantageous in that it provides improved generative models, reinforcement learning techniques, and the combination thereof, to generate compounds in a manner that increases efficiency in the drug discovery process. Specifically, the present invention provides for the design of compounds that are more in line with a target property profile of a clinical candidate for a drug design project such that a time and cost associated with optimising compounds to obtain a clinical candidate is reduced. It will become apparent from the following description the features and combinations of features that provide these advantageous effects.

As an overview, a generative design system and method is described for designing drug-like compounds or molecules that may be candidate compounds for drug discovery projects. In particular, as described in greater detail below, a language model may be used to generate all or part of a compound as character strings, and the language model may be fine-tuned using reinforcement learning to generate compounds that score highly according to a defined reward function.

As will be described in greater detail below, the generative design method includes a so-called ‘prior model’ and a reward function. The prior model may be a language model that has been trained to generate character strings representative of valid, drug-like compounds. The purpose of the language model is to bias the generative search to focus on specific areas of compound space or molecular space that are of interest to explore. The reward function is a function that maps any valid compound to a score. A reward function will typically be defined to be multi-modal, i.e. it will determine a score that balances multiple objectives for a compound that is to be designed, e.g. objectives relating to potency, selectivity, one or more chemical properties, etc.

Again as will be described in greater detail below, the prior model may be stored as a policy (or policy network) for reinforcement learning (RL). At each step of the generative algorithm, a batch of compounds may be generated and then scored using the defined reward function. A reward value may then be attributed to each character in each generated character string representative of the generated compound. The reward values are used to update the parameters of the policy network, in particular to teach the network to increase its probability of selecting good character sequences (i.e. sequences representative of compounds that exhibit desired properties relative to a desired property profile for a drug for a particular project), and decrease the probability of selecting bad character sequences. The policy network progressively learns how to generate compounds that score increasingly highly under the reward function.

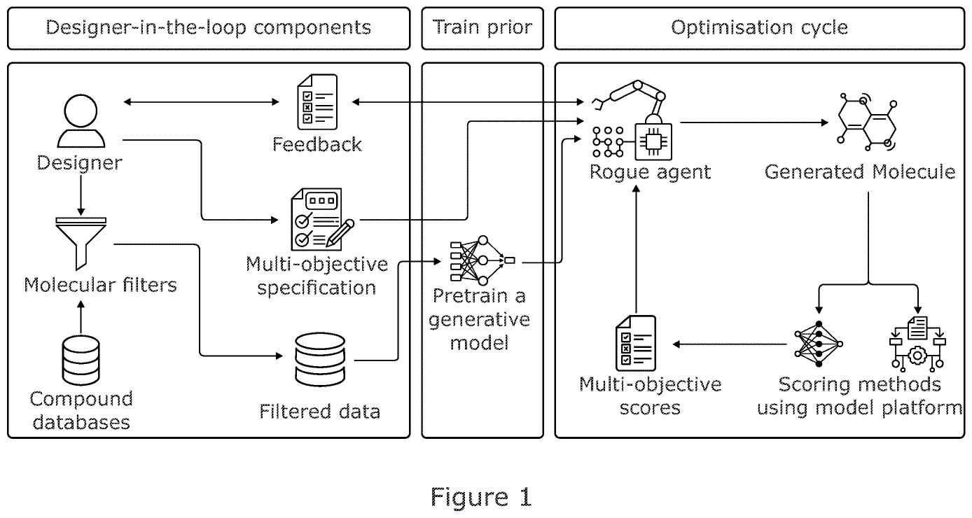

FIG. 1 shows an overview of a pipeline or architecture of a system for the de novo design of compounds in accordance with an example of the invention. As will be discussed in greater detail below, the pipeline includes a generative model for generating sequences corresponding to molecules. The generative model is pretrained using molecular training data. This may be filtered data from one or more molecular filters applied to one or more molecular databases, and may be defined by a user or designer. Depending on the use case, the molecular or compound databases can be activity biased or unbiased. The molecular filters may be used to ensure that the system focuses compound exploration in areas of desirable chemical space, or is filtered to known ‘drug-like’ compounds. The pretrained generative model is used as the starting point of an optimisation cycle to improve the model such that it can generate better molecules in the sense that they are more likely to exhibit more properties that are desired according to the requirements of a particular drug discovery project. In particular, a reinforcement learning agent is used as part of an optimisation cycle to improve the generative model. The RL agent is provided with a multi-objective specification, e.g. specified by a designer, outlining a desired property profile (i.e. a plurality of desired properties) of a compound for a particular drug discovery project under consideration. The RL agent uses this to guide how the pretrained generative model is to be modified with the aim of generating compounds that are more in line with the desired property profile. The RL agent is used to generate a molecule that is then scored according to some appropriate method(s). Specifically, the scoring indicates the suitability of the generated molecule relative to the desired property profile. These determined scores are then fed back to the RL agent, which uses this information to further modify the generative model in a manner that further increases the probability of a molecule being generated that is more in line with the desired property profile. The output from the RL agent may also be fed back to a designer, who can also provide manual input to cause adjustment of the generative model in a desired direction based on what the agent is generating. For instance, active feedback may be used to avoid certain unwanted chemical structures, or certain clusters. A number of iterations of the optimisation cycle may be performed to optimise the generative model for the particular task at hand.

The generation of compounds in this manner has some similarities with language generation, for instance to identify and arrange words for sentence formation. Generative models may be referred to as conditional probability distribution models to reflect the way in which objects—in this case, compounds—are generated based on a learned probability distribution trained on previous data—in this case, relationships between structural features of compounds and biological or other properties, e.g. biological activity against a specific target, of the compounds.

In one example, the generative model is a recurrent neural network (RNN) model or architecture. An RNN model learns an underlying probability distribution over a set of molecular structures. This allows the search for compounds over chemical space to be limited to compounds that may be regarded as reasonable, e.g. compounds that may be readily synthesised.

An RNN is a class of neural network architecture that retains a memory at each step of certain important information from previous steps that may influence a present step. That is, the computation at each step can take into account historical information. In particular, the decision that an RNN made at all previous timesteps t-1, t-2, t-3, etc., will influence the decision to be made at a current timestep t. In that sense, the RNN combines information from the present and the past to determine how to respond to new data. This is useful to extract information from a sequence of data that is found in the sequence itself.

An RNN may be regarded as having a processing unit, cell or node for each time step, where the unit at a given time step is a function of the previous unit corresponding to the previous time step (hence ‘recurrent’) and the current input to the processing unit. The processing unit for the current time step will therefore provide an output at the current time step as well as influence the next processing unit. In this way, the processing units have ‘memory’ of past events which then influence how new data is processed.

In different examples, the generative model may be a different type of model. For instance, a transformer model may be used. A transformer is a deep learning model that can weight input data according to its importance to the task at hand, in particular using the so-called mechanism of ‘attention’ as is known in the art. This mechanism may be useful in terms of scalability to vast amounts of data. A transformer does not need to process sequential input data in order, meaning that greater parallelisation during training is possible.

In order to obtain the trained generative model, the model learns a conditional probability distribution based on target sequences, e.g. on known drug-like compounds. This may typically be done using maximum likelihood estimation (MLE). In particular, in a case in which an RNN is used, the RNN will determine a probability distribution over what the next part of a compound is likely to be, with the aim being to maximise the likelihood assigned to the part that is in fact next in the compound. In particular, back propagation may be used to fit the RNN to the training data, and may be referred to in the art as back propagation through time (BPTT).

Each unit of the RNN may be a long-short-term memory (LSTM) unit. LSTM units can decide which historical information to keep and which to discard. LSTM units can address the issue of vanishing gradients that can be introduced when training an RNN using back propagation. The RNN units may also be a gated recurrent unit (GRU), which is a simplified version of an LSTM architecture that has reduced computational cost. The LSTM-like some other architectures, including GRUs-generates compounds by iteratively selecting a sequence of tokens, e.g. 1-hot vectors. As will be described in greater detail below, each token corresponds to a character, or a sequence of characters, which are added sequentially to the generated compound sequence.

The conditional probability distribution of the trained generative model may be referred to as the prior distribution or, simply, the prior. Once the prior has been obtained based on the provided training data, the trained generative model can be used to generate a compound that is created according to the obtained prior.

In more detail, in order to generate a compound the hidden states of the RNN may be initialised. The RNN is fed with a start token, e.g. in the form of a 1-hot vector, which indicates the beginning of a generated sequence. The model—in particular, the network (e.g. RNN)—receives this token as the 1-hot vector, and uses it to update its internal hidden states. It then outputs a so-called ‘logits vector’ ƒ_{θ}, which specifies the relative probabilities of each choice for the next token. The Softmax function is applied to the logits vector, resulting in a normalised probability vector Ite for the next token in the sequence:

$${π}_{θ} ( {a}_{t} \vert {s}_{t} ) := {Softmax}_{{a}_{t}} ( {f}_{θ} ) = \frac{{e}^{{f}_{θ} ( {a}_{t} )}}{{\sum }_{a} {e}^{{f}_{θ} ( a )}}$$

where aₜ are actions and sₜ are states.

The probability vector is sampled in order to determine the next token in the sequence. The token is passed through the RNN network, such that the hidden states are again updated, and a new logits vector is output from the network. The process is repeated until a token signifying or indicating the end of a generated compound sequence is sampled, or a defined maximum sequence length is reached, thereby finishing the sequence generation. The selected token sequence may then be translated or converted into a character string or sequence (described below) via a defined mapping, where the character sequence is the completed compound or molecular string. A batch of molecules may be generated by parallelising the above procedure, in which case the generation ends when all token sequences are finished.

The prior may be trained to generate compound or molecule sequences similar to those in a training set via supervised learning. A batch of molecule sequences may be converted to tokens, and then passed through the network (model), which outputs a logits vector ƒ_{θ} for each token. In a corresponding manner to above, the Softmax function is applied to obtain a probability vector π_{θ}, which determines how likely it is the current model is selects any given token as the next token. This allows for an evaluation of the probability that this sequence is generated if the current model is sampled. This may be referred to as the likelihood P_{θ} of this batch of data under the current model:

$${P}_{θ} ( {a}_{1} , {a}_{2} , … , {a}_{T} ) = {\prod }_{t = 1}^{T} {π}_{θ} ( {a}_{t} \vert {s}_{t} )$$

The model parameters may then be updated via gradient ascent, so that the so-called ‘log-likelihood’ of the particular batch under consideration is increased:

$${θ}_{n e w} ← θ + α {∇}_{θ} log {P}_{θ} ( {a}_{1} , {a}_{2} , … , {a}_{T} )$$

where α is a defined weight. The process may be repeated to train the model to have a high likelihood of generating sequences in the training set (dataset). Batches of sequences from the training set may be cycled through in order to guard against the model overfitting to only a relatively few compound strings, so as to improve generalisation of the model.

A step of training the generative model may involve hierarchical fine tuning based on different training data in order to obtain the prior for a particular task. For instance, only a relatively small amount of training data may be available for a particular task under consideration. In such a case, training the generative model based on such a relatively small data set may provide a prior not capable of accurately generating new compounds with desired properties. Therefore, training the generative model may involve first training or fine tuning the prior based on a relatively large data set, but which is not necessarily specific to the property profile that is desired as part of the particular project under consideration, e.g. training the prior using a large set of filtered molecules. This prior may then be further fine-tuned based on a smaller data set, but one that is perhaps more specific to the generation task at hand, e.g. to a specific domain. For instance, if the desired target as part of a particular project is a kinase, then the prior trained on a relatively large set of filtered molecules can then be further trained or fine-tuned using an available training set of kinases. Any suitable number of hierarchical training steps may be used to obtain a suitably trained prior, that is both specific enough for the generation task at hand, but retains sufficient knowledge of the greater chemical space to guard against issues such as overfitting.

It may be that a plurality of default or general priors each trained on a relatively large, general data sets (e.g. hundreds of thousands or millions of molecules) may be available. These default priors may be relatively broad and general to drug-like molecular space. The process may then involve selecting one of the available default priors, e.g. the one that is most relevant to the particular drug design project under consideration, and then fine-tuning this default prior using a smaller set of training compounds (e.g. tens of thousands of molecules) that are representative of the type of compounds that are desired to be generated for the particular project at hand, e.g. focused on a specific region of molecular space. It will be understood that more than one round or iteration of fine-tuning may be performed to obtain a trained generative model for generating compounds. For instance, it may be that two steps of fine-tuning, based on progressively smaller and/or more focused data sets, are needed to strike an appropriate balance between obtaining a model that is biased towards generating desirables compounds in a particular region of molecular space versus a model that overfits to a particular region of molecular space.

In order that computational techniques such as the use of a generative model may be used in the context of drug discovery, an appropriate way to represent compound or molecular structures is needed. In particular, appropriate descriptors of compounds are needed such that they may be processed by the computational models. There are various ways in which compounds may be represented for this purpose. For instance, one way of encoding the structure of a compound is by using molecular fingerprints, e.g. extended-connectivity fingerprints (ECFP). These are binary bit strings which indicate the presence or absence of particular substructures in a compound.

One way in which compounds may be represented for use in the present example is via the use of so-called SMILES (simplified molecular-input line-entry system) strings. As is known in the art, such strings are in the form of a line notation for describing molecular structure using short ASCII (American Standard Code for Information Exchange) strings. In particular, SMILES uses symbols to represent atoms and bonds in a compound. A canonicalized SMILES may be used to ensure that each structure has a unique representation where many equally valid versions may be possible. A generative model such as an RNN can learn both the syntax of the SMILES strings as well as the distribution in chemical space that they represent. The sequential nature of the SMILES strings makes them particularly useful in the present context. Some simple examples of compounds and their SMILES representation are: O═C═O for carbon dioxide; CCN(CC)CC for triethylamine; and, c1ccccc1 for benzene.

The trained generative model is used to generate new compounds in accordance with the prior probability distribution that has been learned based on the provided training data, i.e. the training set of compounds used to determine the prior. The generative model generates a sequence representing a new compound by sampling at each time step the next token or character in the sequential molecular representation from the prior.

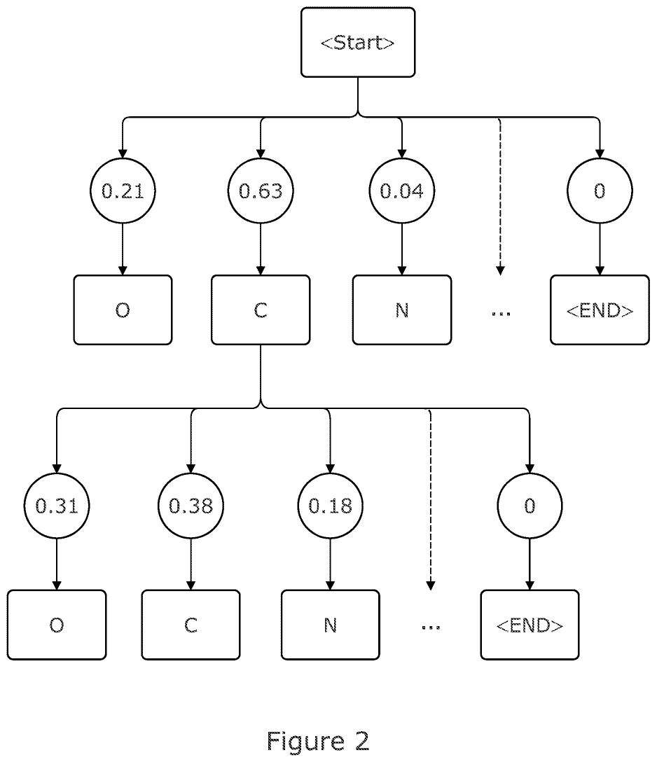

FIG. 2 schematically illustrates how the trained generative model is used to generate a new sequence corresponding to a new compound. A first input token—namely, a ‘Start’ token—is provided, and at the next time step the prior is sampled to determine a first character of the SMILES sequence for the compound being generated. As indicated in FIG. 2, the prior assigns a numeric value to be associated with each of the possible symbols that may be selected as the next character in the SMILES sequence of the compound being generated. In the illustrated example, the symbols O, C and N are shown—representing oxygen, carbon and nitrogen atoms, respectively—each with a numeric value from the prior associated therewith. An ‘End’ symbol is also shown which, if selected, completes the generation of the compound. It will be apparent that only a subset of the possible symbols that may be selected is shown in the figure, for clarity.

In the illustrated example, at the first time step the symbol associated with the highest numeric value from the prior is C, and so a carbon atom may be more likely to be selected as the first part of the generated compound compared with other symbols that have lower numeric values. The generative model then moves onto the next time step to select the next character to be added to the sequence. Again, a numeric value in accordance with the prior is associated with each respective symbol that may be selected as the next part of the compound sequence. At the second timestep, the symbol associated with the highest numeric value from the prior is again C, and so another carbon atom may be more likely to be selected as the second part of the generated compound than other compounds that have lower numeric values. This process continues for successive time steps until the generative model selects the ‘End’ token or symbol. A SMILES string corresponding to the generated compound in accordance with the prior is thereby obtained.

It is desired to use the generative model to generate compounds that have certain desired properties. In particular, a plurality of objectives are defined corresponding to a target profile of an ideal compound for a drug discovery project under consideration, e.g. a target profile for a candidate compound. The properties can be any desired biological, physiochemical, or any other suitable types of properties associated with a compound. For instance, these can include efficacy/potency against a desired target, selectivity against non-desired targets, low probability of toxicity, and good ADME properties.

A mechanism for assessing how good compounds generated by the generative model are relative to the target profile may be provided. Such a scoring function may be any suitably defined function, and it will be understood that many different possible functions are available. As mentioned above, computational models for predicting property profiles of compounds based on their molecular structure may be designed. For instance, machine learning models trained on compounds having known property profiles—i.e. compounds that have previously been synthesised and tested—may be used to then predict the property profile of compounds that have not previously been synthesised, such as the compounds generated by the generative model of the present invention.

In one example purely for illustrative purposes, a reward function or signal for obtaining a score may be defined as: \sqrt{activity_{PARP1}·activity_{CHEK1}}. PARP1 is an enzyme that in humans is encoded by the PARP1 gene, and CHEK1 (checkpoint kinase 1) is a serine/threonine-specific protein kinase that, in humans, is encoded by the CHEK1 gene. The terms activity_{PARP1} and activity_{CHEK1} therefore denote PARP1 and CHEK1 activity scores, respectively, of the generated compound, which are then used to determine the score according to the reward signal defined above. The PARP1 and CHEK1 activity scores may be determined in any suitable manner, for instance by executing suitably trained machine learning models using the generated compound to predict the respective activity scores.

A further example of a reward function may be defined as

$$\frac{1}{2.5} ( {\hat{x}}_{1} + {\hat{x}}_{2} + 0.5 * {x}_{Q E D} )$$

where \hat{x}₁ϵ[0,1] is a probability of pIC50 (i.e. half-maximal inhibitory concentration) being greater than a certain value, e.g. 5.0, against a (first) specific target, e.g. PARP1, \hat{x}₂ϵ[0,1] is a probability of pIC50 being greater than a certain value, e.g. 5.0, against a (second) specific target, e.g. CHEK1, and x_{QED}ϵ[0,1] is a quantitative estimate of drug-likeness. Similarly to as described above, \hat{x}₁ and x₂ may be determined/predicted using a trained machined learning model, e.g. a recurrent neural network (RNN) model. In particular, such a trained RNN may take as input the (known) structural features of the generated compound and map this input to an output of predicted activity. A single trained RNN may predict x₁ and x₂ for a given compound, or separate trained RNNs may be provided for predicting each of x₁ and x₂. x_{QED} is a quantitative drug efficacy assessment, i.e. a metric used to assess how drug like a given compound is, and may be determined as described in ‘Quantifying the chemical beauty of drugs,’ Bickerton et. al, Nat. Chem. 2012, 4 (2), 90-98, as will be understood by the skilled person.

More generally, it will be understood that any suitable number of trained machine learning models may be defined to predict one or more parameters (biological properties), e.g. activity (against a defined target), selectivity (against a defined target), etc., needed for executing the defined reward function. Such machine learning models may be trained based on compounds that have been synthesised and tested previously, such that the relevant parameters, e.g. activity, selectivity, etc., are known/obtained from the testing. The structural features used as input to the machine learning model may correspond to (known) fragments present in the relevant compound, and these may be represented as a molecular footprint suitable for input to the machine learning model. This molecular fingerprint may be an Extended Connectivity Fingerprint (ECFP), e.g. ECFP0, ECFP2, ECFP4, ECFP6, ECFP8, ECFP10 or ECFP12.

It will be understood that any suitable reward function may be defined. Typically, a plurality of different scoring functions/models may be used that each attribute a score to different properties/aspects of a generated molecule. This means that the reward function will be a balance of a plurality of different properties, e.g. the properties defined as part of a target profile of an ideal compound for a particular project. For instance, respective scoring functions/models, or ‘scorers,’ for potency (QSAR model), selectivity, solubility, molecular weight, etc., of a generated compound may be incorporated into the defined reward function. In one example, it may be desired to maximise potency while also controlling the molecular weight and property forecast index of the generated compounds. These reward functions/signals may be referred to as extrinsic reward functions/signals, and may be regarded as providing an indication of the ‘quality’ of a generated compound.

The output from each of the scorers may then be transformed/scaled to a value in a defined range, e.g. between 0 and 1, before being combined to obtain a single reward value/signal that is a combination of each property. That is, the reward function maps any valid compound or molecule to a single score. Different properties may be weighted differently in the overall reward value/signal, e.g. depending on the importance of a particular property. The output from the scorers may be combined using an aggregator that penalises each scaled output based on how far its value is from 1.

The determined score of a compound (e.g. according to the reward signal) generated by the generative model in accordance with the prior probability distribution can then be used to update the generative model. This means that there is a higher probability that the updated model will generate better compounds in the sense that they achieve a higher score relative to the target profile.

It is noted that when attributing reward values to actions selected by the model, the score of the compound indicating how close the property profile of a generated compound is to a target property profile may be regularised by one or more terms in the reward function. For instance, a so-called likelihood-regularisation term may be included that penalises the model for having too high a likelihood of generating a given character sequence. Even if a given character sequence has a desirable property profile, this regularisation term may be used to prevent the model from collapsing to the generation of compounds that are too similar to one another. A further regularisation term, a ‘KL-regularisation’ term, may be incorporated into the reward signal to prevent the model (policy) from straying too far from the prior. The model is in this way encouraged to generate compounds that are not too dissimilar to those that the prior biases towards, e.g. compounds that are known to have drug-like properties.

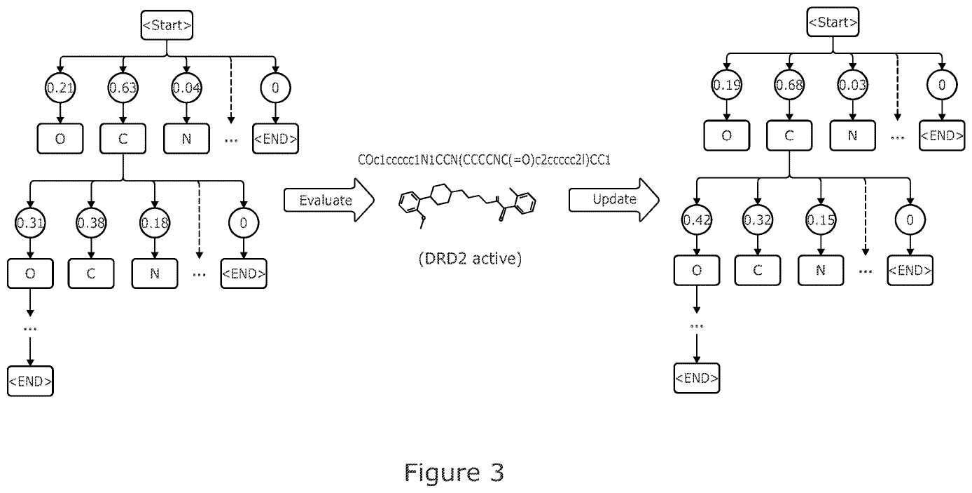

FIG. 3 schematically illustrates how the generative model may be updated to optimise a reward signal associated with compounds generated by the model. As described with reference to FIG. 2, the model assigns numerical values to each of the possible characters (in this example, SMILES characters) that may be chosen as the next token in the sequence. Once a compound is generated according to the model, this generated compound is evaluated by determining a score associated therewith, e.g. using a reward function as described above. The obtained score associated with the generated compound is then used to update the prior probability distribution of the generative model. As illustrated in FIG. 3, this update is in the form of adjusting the numerical values associated with the SMILES characters at each step of the sequence. When the updated generative model is then sampled, the updated numerical values—i.e. updated distribution—result in different (perhaps more desirable) compounds being generated. The particular methods used to update the probability distribution of the generative model are described below.

In the present disclosure, reinforcement learning (RL) techniques are used to update the way in which the generative model makes selections to generate new compounds. RL relates to learning how to map situations or states to actions in order to maximise a numerical reward signal. Expressed differently, RL is a general framework used to teach an agent how to perform some task. In the present context, the state is the compound that has been crafted so far by the generative model. Actions can then be modelled by all the feasible changes that can be made to the current compound. The reward signal can represent the objective or target function, e.g. how ‘drug-like’ the current compound is, how active it is against a specified target, etc.

In RL, an agent selects the actions to take at each step. In particular, the agent has a policy associated therewith that maps a state to the probability of each possible action. Decisions made by the agent are applied inside some defined environment in order to update the environment's state. The RL environment is in particular modelled as a Markov decision process (MDP), meaning that all the information needed to select the next action is provided by the current state, and that there is no added benefit having knowledge of previous states. The reward signal indicates how beneficial it was to take a certain action when in a certain state, and a long-term return of cumulative rewards over time may be determined. For a SMILES representation of compounds, it may only be sensible to consider the reward of complete compounds against the objective profile. The task performed by RL is, given the rewards obtained by taking certain actions in certain states, to update the policy of the agent so that an expected return is increased, i.e. compounds that are more in line with the target profile are generated. That is, the reward signals are used by the agent to update its policy, changing how it performs its decision making. In the present context, the task is to generate the best possible candidate compound or molecule. The agent executes actions in the form of selecting a token that corresponds to a character in the compound string, and the environment simply keeps track of the current state, which is the character string generated thus far. The environment is therefore treated as being the full character string generated thus far (which is then featurised by the RNN), and hence it provides all necessary information for selecting the next token (meaning that it is an MDP). The reward signals for each action are determined using the compound or molecules scores returned by the reward function.

In the present example, the prior probability distribution of the generative model can be regarded as the initial or starting policy of the RL agent, and may be referred to as the prior policy. The aim is therefore to finetune or update the prior policy in a manner that produces compounds that maximises the defined scoring function. Whereas the network using the prior policy may be referred to simply as the prior, the network that has since been modified from the prior using RL may be referred to as an agent policy. Like the prior, the agent is therefore also a generative model—e.g. an RNN—with the same architecture as the prior. The RL agent is used to iteratively update parameters of the current policy network to generate higher-scoring compounds or molecules according to the defined scoring function.

The task to be performed by the agent may be referred to as episodic in that it has defined start and end points, i.e. the start and end of a generated sequence representing a generated compound. Like described above, the end of the task occurs when the ‘End’ token is sampled by the agent, indicating a complete compound has been generated.

The sequence of actions A=a₁, a₂, . . . , a_{T} for each episode represents the SMILES characters that are generated, and the product of the action probabilities

$$P ( A ) = {\sum }_{t = 1}^{T} π ( {a}_{t} \vert {s}_{t} )$$

represents the model likelihood of the generated sequence, the likelihood being a measure of how well the model fits to the sampled data, i.e. the generated sequence, where π is the agent policy (or current policy) of actions at over states sₜ.

For a scoring function S(A) that rates the generated sequences relative to a desired profile, the aim is to update the agent policy π from the prior policy πₚ in a manner that increases the score of generated sequences. As the prior policy has been designed and trained in a way that learns the syntax of the SMILES strings and a distribution of molecular structures, then it may be preferable to determine the agent policy π as an update that is anchored to the prior policy πₚ so that this learning is not lost.

An augmented likelihood P(A)_{U} may therefore be defined as a prior likelihood modulated by the desirability of a generated sequence based on the scoring function:

$$log {P ( A )}_{U} = log {P ( A )}_{p} + σ S ( A )$$

where σ is a scalar coefficient.

A return G(A) of a generated sequence A may in this case be regarded as the agreement between the agent likelihood log P(A)_{A} and the augmented likelihood log P(A)_{U}:

$$G ( A ) = {[ log {P ( A )}_{U} - log {P ( A )}_{A} ]}^{2}$$

An aim of the agent may then be to learn a policy that maximises the expected return by minimising a cost function L(θ)=−G, where θ is one or more parameters of the generative model, e.g. an RNN. That is, the parameters of the model are updated to maximise the expected return, optionally subject to an entropy regularisation term to prevent collapse. Specifically, this is repeated to update the network parameters in an iterative manner so that the network becomes more likely to generate compounds with high rewards.

There are different available methods for updating or optimising a current policy of an RL agent. One example is policy gradient methods, which rely upon iteratively optimising policies directly relative to an expected return by gradient descent, e.g. by minimising a suitable loss or cost function. This class of methods includes so-called ‘vanilla’ policy gradient methods and trust region/natural policy gradient methods, for example. Vanilla methods can have poor data efficiency and robustness, and trust region methods can be relatively complicated, computationally expensive, and difficult to tune.

One method that can achieve data efficiency and high performance while only using a first order optimisation scheme is so-called proximal policy optimisation (PPO). Whereas standard policy gradient methods may perform only one gradient update per generated compound, an objective function or target profile may be defined that allows for several epochs of optimisation on the sampled data that are compatible with stochastic gradient descent. In particular, this ‘surrogate’ objective function—and loss function—includes clipped probability ratios that forms a pessimistic estimate—i.e. lower bound—of the performance of the policy. The PPO method is similar to a trust region method in that constraints on the magnitude of policy updates are set to avoid poorly-chosen actions to impact too severely on training a policy. The PPO method aims to enable the biggest possible improvement in a policy at each step without changing so much that policy performance collapses (which is an issue with some previous methods).

Another technique that may be used to improve the performance of a policy gradient method—that can be used in combination with the clipped probability ratios of PPO—is KL (Kullback-Leibler) loss or divergence. As is known in the art, KL divergence is a measure of how two probability distributions differ from one another. In a similar manner to the use of clipped probability ratios, application of a KL divergence technique can be used to ensure that updates to the agent policy do not diverge too much from a prior policy. In particular, the loss function may be modified to introduce or adapt a penalty coefficient such that a large value of KL divergence between the current policy and the previous policy is penalised.

The inclusion of a KL divergence term into the loss function particularly addresses the issue of the learned policy collapsing in terms of the validity and/or uniqueness of the generated compounds. The KL divergence is calculated between the prior policy and the learned agent policy. In this way, the model is incentivised to not deviate too far from the prior policy (in order to keep the desirable properties or features of the prior policy).

A further technique that may be used to improve the policy gradient method is to introduce a degree of regularisation to the likelihood function. Again, this can involve the inclusion of a further term in the loss function that is to be optimised (minimised). Typically, this may include a penalty term that penalises complexity of the function to be predicted, i.e. the policy to be optimised.

Another technique that may be employed to improve the policy gradient method is to include a measure of maximum mean discrepancy (MMD). Like KL divergence, MMD refers to a measure of distance between two different probability distributions. The inclusion of MMD encourages policies that lead to different state visitation and/or action choice distribution compared to previous policies. In particular, the agent may learn to actively avoid areas of chemical space that are already known, meaning that exploration of novel chemical space is obtained. This may be regarded as negative feedback during the policy optimisation in the sense that a set of compounds are defined that span part of chemical space that should be avoided, even if they score highly on the reward function. Iterative use of this diversity-inducing term into a policy gradient method allows for distinct policies to be sequentially obtained.

As part of such a technique, an MMD metric is used to measure the difference between two trajectory distributions. The metric may be an integral probability metric that measures the difference using test functions from a function space such as a unit ball in a reproducing kernel Hilbert space (RKHS) defined by a kernel. In the described example, this kernel of similarity between distributions may be defined to be a Tanimoto similarity between molecular fingerprints. Such a kernel is better suited to the present context of molecular generation, i.e. the operating on chemical space. This is also beneficial as each fingerprint is unique for each compound.

The loss function used as part of the executed policy gradient method may therefore include terms relating to one or more of: standard PPO loss (clipped probability ratios); KL divergence to the prior policy; likelihood regularisation; and MMD. It is found that inclusion of terms associated with more of these techniques leads to better performance in terms of policy optimisation.

The policy gradient method may also include a scheme for reducing the variation in the method, in particular using generalised advantage estimation (GAE). In RL algorithms, one issue is the delay between actions being taken by an agent and their (positive or negative) effect on rewards. Value functions are used to estimate how good an action is before the associated award materialises. For parametrised stochastic policies, gradient estimates of the expected total returns may be obtained. The variance of such gradient estimators can scale unfavourably with time, as the effect of an action is confounded with the effects of past and future actions. For actor-critic policies, lower variance estimators may be defined at the cost of introducing bias. GAE can be used to reduce variance and bias in this context. In particular, GAE introduces an extra hyperparameter that blends a temporal difference update (with higher bias and lower variance) together with a Monte Carlo update (with higher variance and lower bias) in order to provide an overall reduction of variance and bias. Specifically, more stable learning curves during policy optimisation may be obtained with the use of GAE.

The policy gradient method may be defined to include a greater amount of feedback in terms of reward signals. This means that instead of receiving a single reward signal after an entire compound has been generated, reward values can be assigned to smaller parts of a compound rather than one reward for an entire compound. In particular, a level of importance may be attributed to features that the predictive model finds to be the most correlated with a certain prediction, e.g. high activity. This therefore allows for certain parts or sub-sequences of a SMILES string, for instance, to be identified as more important than others so that the generative model learns which specific action sequence is the most important for a specific result. The use of such a ‘dense’ reward signal can increase accuracy relative to the ‘sparse’ reward signal for each completed compound, as the agent can learn to explore more efficiently around well-scoring motifs. Dense rewards signals may in particular be obtained by using integrated gradients, which is a technique for attributing an importance value to each input feature of a machine learning model based on the gradients of the model output with respect to the input.

The described generative model may be used for any of: generating full compounds; generating R vectors attached to a fixed structure (referred to as core fixing); and, core replacement, in which just the core structure with attachment points is generated. In a case in which full compounds are being generated, the agent starts with an empty string and generates the compounds token by token by sampling the current policy until the ‘End’ token is sampled.

In certain cases, it may be that certain defined molecular cores are identified as being of interest and that novel compounds are generated while still retaining such an identified core. Applying such constraints in the generation process may be beneficial or necessary to retain certain desired properties associated with certain cores, or to explore a certain region of chemical space that is of interest or is relevant. This therefore reduces the probability of generating compounds that are not of interest for some reason, e.g. they are not feasible.

In an example of the present invention, the framework to perform policy optimisation is adapted to incorporate core fixing. In particular, a core fixing algorithm may be incorporated into a PPO approach. Whereas using core fixing in an RNN sampling technique, for instance, would involve sampling an already-trained model to add to the fixed core, when using core fixing with PPO the model needs to be trained through the PPO loss. With core fixing, two types of policies are included in the method: a core policy; and an agent policy. The core policy is deterministic and is fully defined by the representation of the (fixed) input core. The agent policy is only played/sampled when a point in the core policy is reached where it is required to start inserting R vector tokens, where R represents the non-fixed core part of the compound being generated.

During the optimisation process, it is the agent policy that is to be updated and learned (whereas the core policy is not updated). To train the PPO algorithm and propagate updates correctly, a policy mask may be tracked through the process. In general, a policy mask is for filtering out impossible or unavailable actions. For instance, the policy mask may be a binary tensor where 1 indicates that the agent policy action was played and 0 means that the core policy action was played. Policy updates are then only calculated for unmasked values. This is also the case in the backpropagation step. This would be in contrast to using core fixing during a sampling only process, in which updates would be performed using all actions sampled across a compound generation episode, meaning that mask tracking would not be needed.

Core fixing may also be incorporated into examples in which GAE is implemented as part of the PPO algorithm. The policy mask may also be tracked in this case. The GAE advantages and returns may then only be calculated on the unmasked values.

In an example in which compounds are being represented as SMILES strings, a defined or fixed core may be regarded as a partial or incomplete SMILES string with open positions where R vectors are to be chosen and inserted by the generative model. For instance, one, two, three, or more open positions (R-group attachment groups) may be included. In such an example, the generative model may be constrained to follow the fixed core (e.g. by sampling the core policy) at each step until it reaches a point in the sequence corresponding to an open position. At this step, the generative model samples the agent policy as described above to generate a molecular fragment that is to be next in the sequence of the compound being generated. In an example of the invention, the particular position in a molecular sequence at which an R vector is to be inserted may be selected. This may be in contrast to the algorithm being left to decide where to insert an R vector in a molecular sequence. This may improve the feasibility of molecules being generated.

In certain examples where a core is to remain fixed, the generative model may not be trained on a specific core. Instead, different R-groups in a training set of compounds may be identified and isolated, and the model may then be trained on these R-groups. This means that the model may not actually see the (fixed) core itself.

In examples in which more than one R-group is to be generated for attachment to a fixed core, the ordering of the R-groups in the character sequence representation of the compound should not influence the generation of R-groups. If a compound in the training set includes two R-groups (A and B) for instance, then it may be desired to include two representations of the compound in the set on which the model is trained, in particular a first representation in which R-group A appears before R-group B in the sequence, and a second representation in which R-group B appears before R-group A in the sequence.

In a case in which the cores are being replaced, the aim is to only generate a core structure of a compound and, depending on the particular use case under consideration, it may be conditioned on the R vectors that are to be attached to the generated core. A separate prior may be trained for that particular use case. In a case in which just the core is being generated, a prior may be trained only on core structures. For instance, if the compound with the SMILES string c1ccc&1cc1.CC&1 is included in the training data, then only the core element of this—specifically, c1ccc&1cc1—would be used for training. Separately, a prior may be trained where fragments are put in front of the core. In the above example, the SMILES string c1ccc&1cc1.CC&1 would be transformed to CC&1.c1ccc&1cc1 and this prior would allow for cores conditioned on the R vectors to be generated. This may be useful in cases in which only a relatively limited amount of data is available, e.g. early-stage projects, and the properties of a couple of different cores may be synthesised and checked to find out what the series definitions for a given target protein should be, for instance.

In more detail, in examples in which a core is to remain fixed, a prior may be trained to generate valid R-groups rather than complete compounds or molecules. However, a reward function will be defined that scores a complete compound, as above. A particular token may be used to indicate where a generated fragment should be attached to the fixed core, and a further token may be used to break up the R-groups generated by the model if more than one R group is being generated to attach to a given fixed core. Different priors may be trained in dependence on the number of R-groups to be generated. Once an R-group sequence (including one or more R-groups, as needed) has been generated, the (fixed) core and generated R-groups may be merged to create a complete compound or molecule. The complete compound is scored via the defined reward function, and this score is used to update the (current) policy network as described above, thereby training the policy network to generate R-groups that result in increasingly higher-scoring compounds, i.e. compounds that have a property profile that is closer to a target property profile (for a candidate compound).

In the above, molecule representation using SMILES strings is described for use in generating new molecules using the described techniques. In examples of the invention, different molecule representations may be used. For instance, a so-called CHUCKLES representation of molecules uses monomer symbols in a corresponding manner to how atomic symbols are used in SMILES, and was first described in “CHUCKLES: A Method for Representing and Searching Peptide and Peptoid Sequences on Both Monomer and Atomic Levels”, Siani et al., J. Chem. Inf. Comput. Sci., 1994, 34, 588-593. CHUCKLES was developed to infer SMILES of polymers from their sequences, and vice versa. In this way, CHUCKLES may be regarded as an extension to SMILES. In particular, ring closures are interpreted on a monomer-definition basis (external or unmatched ring closures are possible), and disconnected fragments may be merged as they are fed into a model (left to right).

The use of CHUCKLES representation may allow a molecule to be evaluated multiple times during the generation process, and not just at the end of the process for a completed molecule. This means that more frequent feedback may be given to the model, which in turn may lead to faster optimisation of the model. In particular, with CHUCKLES the notation is such that partially generated molecules may still be evaluated. Considering a benzene ring with a small carbon chain, the CHUCKLES representation may be c1ccc&1cc1.CC&1. Here, the &1 denotes an attachment point, and the dot separates the core structure of the molecule from the fragments attached to the core. In this example, therefore, c1ccccc1 is the core, and CC is the attached fragment, where & 1 indicates how the two connect together.

This notation therefore allows for more frequent feedback in the generation process. In particular, as it is known where a core ends and where a fragment begins—i.e. via the use of a dot—then it is known that whenever a dot is encountered in the generation process, a valid molecule fragment has been generated. Hence, in the above example, when generating a CHUCKLES string for a benzene ring with a small carbon chain, once the string c1ccc&1cc1. has been generated, it is known that evaluation of the molecule fragment generated thus far may be performed and fed back to the model. In particular, it is known that &1 and the dot may be removed to evaluate c1ccccc1. Then, once the full string has been generated the completed molecule can also be evaluated.

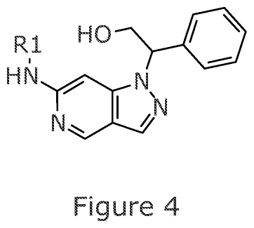

In an example in which core fixing is used, the use of CHUCKLES can make the way in which the core structure is provided to the generative model more intuitive. This is explained with reference to the molecule illustrated in FIG. 4. In one example, the molecule illustrated in FIG. 4 is provided, and it is desired to generate different designs for the R1 vector using the generative model. With SMILES notation, the molecule would be represented as a SMILES string, and it would need to be indicated to the model at what point to insert tokens for the R1 vector. In particular, the core and R1 vector may be represented by c1ccc(cc1)C(CO)n2c3cc(NR1)ncc3cn2.

The core fixing may then be implemented using the core and agent policies as described above. In particular, if a core argument is provided then the generation process is started by rolling out the core policy. This simply means that the tokens from the SMILES core are placed sequentially at each step of the generation process. This continues until an R symbol (for the R1 vector) is encountered in the sequence. The idea is that the R1 vector is replaced with the tokens sampled by the generative model according to the agent policy. The agent policy is played/sampled until the agent decides to sample an extra closed parenthesis symbol, indicating the end of the R1 vector. This signals to the model to switch back to the core policy until it encounters the next R symbol, or the ‘End’ symbol is sampled, in which case the generation process or episode ends.

The issue with the above is that at the time of sampling a given token, the agent is only aware of—and the agent policy is conditioned on—the tokens that were previously placed in the sequence, but not the token that will be placed later in the sequence. In particular, for core fixing the issue is that, while it is known at the start what core tokens will come after R1, the agent does not know this.

Returning to the above example, the agent will see that the tokens placed so far by the core policy form the string c1ccc(cc1)C(CO)n2c3cc(N. Through the agent policy, the model may decide to close one of the rings by placing some atoms followed by a 3. Normally, this could result in a valid SMILES string, but not in this example because the remaining core tokens also close the same ring, i.e. the remaining core tokens are) ncc3cn2. The result of such a generation episode would therefore be an invalid molecule.

It is therefore the case that the way in which a SMILES string with an R vector is represented will result in different success rates of the training process. In particular, some representations make training more difficult than others. In the above example, a better representation—and input to the model—would be c1cc(ccc1C(CO)n2c3cc(ncc3cn2)NR1). This is because the agent policy cannot break any rings, and overall the episode rollouts with this core representation would be more similar to the prior policy the agent already has knowledge of. Nonetheless, it may not be apparent to a user what would constitute a good or a bad core representation, and so it is in this sense that use of CHUCKLES may be chosen in certain examples.

With CHUCKLES, the above issues are readily overcome because the attachment (R1 vector) will always fall at the end of the string. As such, the full context of the core molecule will be given to the agent, and the attachment point just needs to be specified using the &1 notation. Returning to the above example, the core may be represented as either c1ccc(cc1)C(CO)n2c3cc(N&1)ncc3cn2 or c1cc(ccc1C(CO)n2c3cc(ncc3cn2)N&1) without significantly affecting the performance of the generative model.

An extra step may be needed if using the CHUCKLES representation in the above generative model. In particular, the training data which is used to determine the prior may need to be transformed from the more commonly-available SMILES representation. To do this, each molecule in the training set may be split into the core structure and fragments attached to it, and then pieced together in the CHUCKLES notation.

In examples of the invention, the described model and method advantageously uses a so-called SELFIES (SELF-referencing Embedded Strings) syntax to represent compounds, as described in “Self-Referencing Embedded Strings (SELFIES): A 100% robust molecular string representation”, Krenn et al., Machine Learning: Science and Technology, 2020, Vol. 1, No. 4. Unlike the SMILES notation, each SELFIES string corresponds to a valid molecule/compound, and SELFIES can be used to represent every molecule. Moreover, SELFIES may be applied to directly to machine learning and generative models without adaptation of the models, with each generated compound being a valid compound. Broadly, in SELFIES information relating to branch length and ring size is stored, which assists in preventing the possibility of invalid syntactical strings, e.g. a string with more opening brackets than closing brackets.

An advantageous feature of examples of the described method and system is that it can be used to generate compounds that are both of high quality and diverse. In some examples, a single mode of relatively high reward may be found by the model, and then the model focusses on generating molecules close to this node. It is desired to perform a more global optimisation of molecular space (for a particular project) to generate a more diverse range of compounds that score highly relative to a particular target profile. In general terms, this is achieved by scoring a generated compound not only on how close it is to a defined target property profile, but also on how dissimilar it is to other compounds that have been generated (or other compounds in a training set), where a higher reward is given the more dissimilar the generated compound is.

A process for generating more diverse compounds may be performed as follows. A prior may be trained as outlined above based on a defined training set of compounds, e.g. that have drug-like properties. The prior may be sampled to generate a batch or set of one or more compounds. The generated compounds may be scored according to a ‘standard’ (extrinsic) reward function defined as outlined above. The prior may then be modified based on the determined compound scores using the RL agent, e.g. using a PPO method. In particular, the parameters of the generative model, e.g. RNN, may be updated to increase the probability of the model generating high-scoring compounds according to the defined reward function. That is, the prior probability distribution is modified to obtain an updated or current probability distribution. This process may be repeated for several cycles or iterations to generate a plurality of sets/epochs (e.g. fifty, or any other suitable number of sets) of compounds by sampling the current probability distribution.

After a sufficient or desired number of compounds have been generated (e.g. several thousand compounds), a defined number of the top-scoring generated compounds are identified (e.g. the top 500-1000 compounds) and stored. These top-scoring compounds may then be used to train a Gaussian Process (GP) model to predict or approximate the compound scores (using the defined (extrinsic) reward signal) as a function of the structural features of said compounds. A GP model is used to model an objective function which, in this case, describes a relationship between structural features of a compound and its score according to a defined reward signal. The model may be a Bayesian model, where a surrogate function of the model may be a Gaussian Process.

As well as predicting the value of the objective function, a GP model also generates a measure of uncertainty, i.e. a way to quantify how certain the model is of its own prediction. A standard deviation may be determined as the predictive uncertainty of the model output. Typically, a GP model may be less certain of a prediction at a point of the objective function the further away it is from evaluated/observed points in the functional space. In this way, the measure of uncertainty generated by a GP may be used as an indication of how dissimilar a compound being evaluated by the GP model is relative to the compounds used to train the GP model. In particular, the more uncertain the GP is of its own prediction for a given generated compound, the more dissimilar said generated compound may be regarded as being relative to the compounds used to train the GP model, i.e. the top-scoring compounds generated thus far. The measure of dissimilarity between compounds may therefore be regarded as how far away compounds are from one another in molecular space.

A new or further reward function may then be defined that aggregates the compound score from the (original) defined reward function (that scores a compound based on its proximity to a target property profile) with a second reward term/function that scores a compound on how dissimilar it is to the top compounds (i.e. highest-scoring compounds) generated thus far. While the original reward function may be regarded as providing an ‘extrinsic reward’ (as described above), the second reward function may be regarded as providing an ‘intrinsic reward’. The new/further reward function may be defined as a sum of the original (extrinsic) reward and second (intrinsic) reward, where the second reward may be weighted relative to the original reward before being summed together. In this way, the importance of dissimilarity relative to achieving a high score (relative to the target property profile) can be tuned as desired. This aggregated/further reward function therefore rewards the model for generating compounds that are both high-scoring and dissimilar to compounds that the model has generated thus far, thereby leading to the generation of new modes, i.e. the generation of compounds in molecular space that has not yet been explored. This therefore allows for more global optimisation of molecular space when generating compounds that may become candidate compounds for a particular drug design project.

A new batch or set of one or more compounds may then be generated by sampling the current policy of the generative model, with these compounds being scored according to the aggregated/further reward function. Note that this requires the new generated compounds to be evaluated by the GP model.

The GP model may then be retrained based on the best compounds generated thus far. That is, the (top) set of compounds—including the top x highest-scoring compounds, e.g. top 50, 100, 200, 500, 1000 scoring compounds, or any other suitable number—may be determined again (which may now include one or more of the most-recently generated compounds), and the GP model is trained based on the updated top set.

This process may be repeated iteratively to generate several batches of compounds, with the GP model being fitted to the best compounds generated across all runs/iterations so far.

An example of determining the second (intrinsic) reward signal for a given compound is as follows. As mentioned above, this intrinsic reward is based on the uncertainty value associated with the given compound output by the GP model. In particular, the uncertainty value may be normalised to be in a specific range, e.g. [−1, 1]. In order to perform this normalisation, the prior (or current) probability distribution of the generative model may be sampled a prescribed number of times, e.g. several thousand (such as 5000), to generate ‘normalisation’ compounds to be used to calculate normalisation constants. Specifically, the uncertainty value of each of these generated ‘normalisation’ compounds is determined using the GP model, and median and maximum values of the uncertainty are determined based on these obtained uncertainty values. The determined median and maximum values of the uncertainty are used to normalise a (further) generated compound to be in the specific range, and then this value may be further scaled based on the mean extrinsic reward (i.e. mean value of the compound score) of the compounds used to train the GP model, i.e. the ‘best’ compounds (as described above).

A specific example of the second (intrinsic) reward function is as follows. Let

$$D = {\{ {m}_{i} \}}_{i = 1}^{1000}$$

be the set of compounds used to train the GP model, i.e. the top 1000 scoring compounds (according to the compound score or extrinsic reward function) from an initial run (e.g. several cycles or iterations) of the generative model with the prior/current probability distribution. Then, let

$$C = {\{ {m}_{k} \}}_{k = 1}^{5000}$$

be a set of 5000 compounds randomly sampled from the prior/current probability distribution. It will be understood that the size of the example sets used here, i.e. 1000 and 5000 compounds, respectively, may be selected to be any suitable size in different examples. The second (intrinsic) reward function \hat{r}(m) may then be defined as follows

$$\hat{r} ( m ) = β * min \{ max \{ \frac{{σ}_{G P} ( m ) - M}{S} , - 1 \} , 1 \}$$

$$where$$

$$M = {median}_{{m}_{k} ϵ C} ( {σ}_{G P} ( {m}_{k} ) )$$

$$S = {max}_{{m}_{k} ϵ C} ( {σ}_{GP} ( {m}_{k} ) - M )$$

$$β = 0.1 * {mean}_{{m}_{i} ϵ D} ( r ( {m}_{i} ) )$$

That is, M and S are the median and maximum values of the uncertainty values of the randomly sampled set, and β is a scaled value of the mean of the extrinsic reward (compound score) of the top-scoring set.

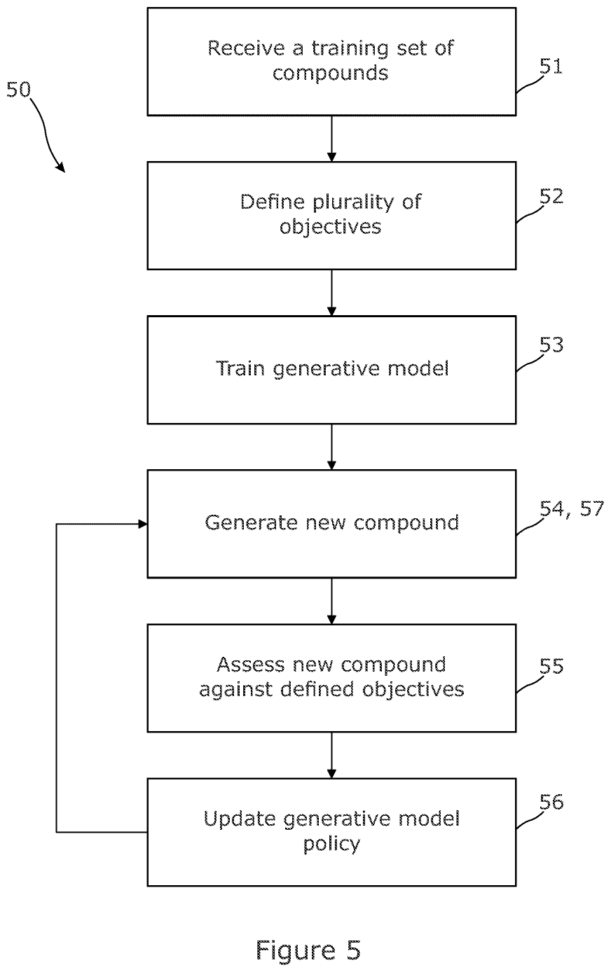

FIG. 5 summarises the steps of a method 50 for de novo drug design in accordance with the invention and using the architecture illustrated in FIG. 1. At step 51 of the method 50, a training set of a plurality of training compounds is received or defined. Each training compound has a plurality of structural features. The training set can be defined or acquired in any suitable manner, e.g. via known computational methods and/or with human input. In one example, a starting group of compounds (including millions of compounds, for instance) may be reduced in number by applying known methods to keep certain ones of the compounds with at least some desired features for a particular project at hand. One or more filters may be applied to the retained compounds to remove any undesirable compounds. The filters can be defined according to any appropriate criteria for selecting (or filtering) desirable compounds from undesirable compounds. For instance, one useful filter may be adapted to remove duplicate compounds. Another filter may be adapted to remove compounds having a certain level of toxicity. The filtered set of compounds may then form the training set. The training set may include compounds that have previously been synthesised and tested experimentally such that one or more of their biological or other properties are known.

At step 52 of the method 50, a desired property profile is designed that is indicative of one or more desired properties of a drug (drug compound) to be designed. The desired properties can for instance include a desired level of: activity against a specified target; selectivity against a specified target; toxicity; absorption; distribution; metabolism; and, excretion. The desired properties may include biological, physiological and/or pharmacological properties, e.g. biological, physiological and/or pharmacological activity.

At step 53 of the method 50, a generative model is trained using the training set to obtain a prior probability distribution of the structural features in the training set. In some examples, the method 50 may simply involve receiving or acquiring a generative model that has previously been trained, such that the steps of training the generative model do not need to be included explicitly as part of the inventive method. The generative model may for instance have been previously trained on a publicly available compound database.

In one example, the training set includes a first training set of a first plurality of training compounds, and a second training set of a second plurality of training compounds, where the second training set is different from the first training set. In such a case, training the generative model may involve using the first training set to obtain a first prior probability distribution of the structural features in the first training set, and using the second training set to modify the first prior probability distribution to obtain the prior probability distribution. In this case, the prior probability distribution incorporates a distribution of the structural features in the second training set. In this way, the generative model may be trained in a hierarchical manner to obtain the prior. For instance, the first training set may include a relatively large number of (filtered) compounds, with the second training set including fewer compounds, but compounds that have at least some desired properties of an ideal compound. The first training set may include training compounds that are from a certain region of molecular space that is known to have compounds that have drug-like properties. The second training set may then include training compounds from a sub-region of this region that includes compounds that have certain desirable properties for the particular drug design project under consideration. In this way, the prior is trained in a manner that increases the probability of generating compounds having desired properties, but is also trained in a manner so as not to overfit to relatively few, specific compounds.

At step 54 of the method 50, an initial set of one or more compounds is generated using the generative model, in particular by sampling the prior probability distribution. In particular, this may involve sequential sampling of the prior to obtain a string of tokens representing the initial compounds. The compounds are represented in an appropriate manner such that each token in the string may represent an atom, fragment, bond, etc. of the generated compound. For instance, each compound may be represented in SMILES, CHUCKLES or SELFIES notation, as outlined above. In certain examples, SELFIES notation may be preferred as this advantageously guarantees the generation of valid compounds. The prior may be sampled until an ‘End’ token is sampled, indicating that the generation process is complete.

At step 55 of the method 50, a compound score associated with each of the respective compounds in the initial set is obtained. The compound score is indicative of the desirability of each generated compound for a particular task at hand. For instance, the score is indicative of a proximity of a property profile of the generated initial compound to the defined desired property profile, i.e. a combination of desired values or ranges for respective properties.

The compound score may be determined in any suitable manner. In one example, a defined reward function is used to evaluate the generated compound. A relatively high value of the reward function may indicate that the generated compound is of relatively high desirability. In an example, the compound score may be determined using a machine learning (ML) model that is trained on structure-activity relationships of a population of (previously synthesised) compounds such that the model predicts an activity of the generated compound based on its structural features, e.g. as defined by the string of tokens representing the compound. The score—whether it be in the form of a reward function output and/or ML model—may be a one-dimensional value (e.g. between 0 and 1) indicating an overall desirability of the compound. In this way, the method—which will use the obtained score to update the model—will be unaware of how various parts of the compound are aggregated to obtain the 1D number. In different examples, different distributions may be determined for different ones of the plurality of desired properties/objectives to be achieved in an ideal compound.

At step 56 of the method 50, a current policy of the generative model is determined. The current policy is determined by modifying the prior probability distribution based on the determined/obtained compound score using a reinforcement learning algorithm. In particular, the idea is that the prior is modified in a manner that a next set of one or more compounds generated according to the determined distribution will have a higher compound score than the previously-generated set of compounds, i.e. the next set of compounds is more desirable than the previous set of compounds according to the desired property profile. Modification of the prior can involve updating the parameters of the generative model, e.g. parameters of an RNN.

At step 57 of the method 50, a new set/batch of one or more compound is generated in accordance with the current policy. In a corresponding manner to above, the method may then subsequently involve determining the compound score associated with the generated new compounds, and updating the current policy based on the determined compound scores associated with the respective generated new compounds. Indeed, the steps of generating new compounds based on the current policy, determining the scores associated with the new compounds, and updating the current policy based on the determined scores may be repeated in an iterative manner. The aim may be regarded as being to optimise the score associated with the generated new compounds, indicating optimised desirability of said compounds relative to the defined desired property profile. The steps may be repeated for a prescribed number of iterations, until the compound score satisfies a threshold condition, or until any improvements in the score across iterations begin to level off, for instance. As mentioned, it may be the case a batch of a plurality of new compounds are generated at each iteration, with the associated scores being used to perform a single update to the current policy of the generative model. In addition, a replay buffer may be used, in which the generated molecules of the most recent batches (e.g. most recent N batches, where N is a positive integer) are stored/added to the replay buffer, and may be used more than once to update the current policy.

In one example, the reinforcement learning algorithm is a proximal policy optimisation method or algorithm. In addition to a term in the associated loss function relating to proximal policy optimisation, terms relating to one or more of a Kullback-Leibler divergence, a likelihood regularisation, and a maximum mean discrepancy may be included in the loss function. In one example, terms relating to each of these are included.

In some examples, the score attributed to, or associated with, a generated compound is not limited to indicating its proximity to the desired property profile. Instead, as described above, the score may be indicative of a combination of a compound's proximity to the desired property profile (where a higher score is attributed to a compound that is closer to the desired property profile) and of how similar it is to other generated compounds (where a higher score is attributed to a compound that is less similar to other generated compounds). The dissimilarity of a generated compound may be determined via the use of a Gaussian Process model as described above, where the GP model's uncertainty in its prediction of the compound score of a compound (i.e. its proximity to the desired property profile) is used as an indicator of the compound's similarity. An overall or further compound score that aggregates the compound score and dissimilarity score in any suitable way may then be used to update the generative model's current policy.

Certain ones of the generated compounds may be selected for synthesis to ascertain their properties via experimentation. This selection may be based on which compounds have the highest determined compound score (or further compound score). The generated compounds may be scored by a machine learning (ML) model that predicts properties of the compounds as a function of their structural features. The compounds that are predicted to have desirable properties relative to a defined property profile according to the ML model may be selected for synthesis.

Generated compounds that have been synthesised to ascertain their properties may be used to assist with further iterations of the drug design process. The synthesised compounds may be added to the training set used to train the generative model, with the model being retrained to obtain an updated prior that is then sampled to generate a further batch of compounds, thereby increasing the probability of desirable compounds being generated. The synthesised compounds may be added to a training set of compounds used to train the ML model such that the accuracy of the predicted properties of compounds executed by the ML model increases, thereby better informing with further compounds to select for synthesis.

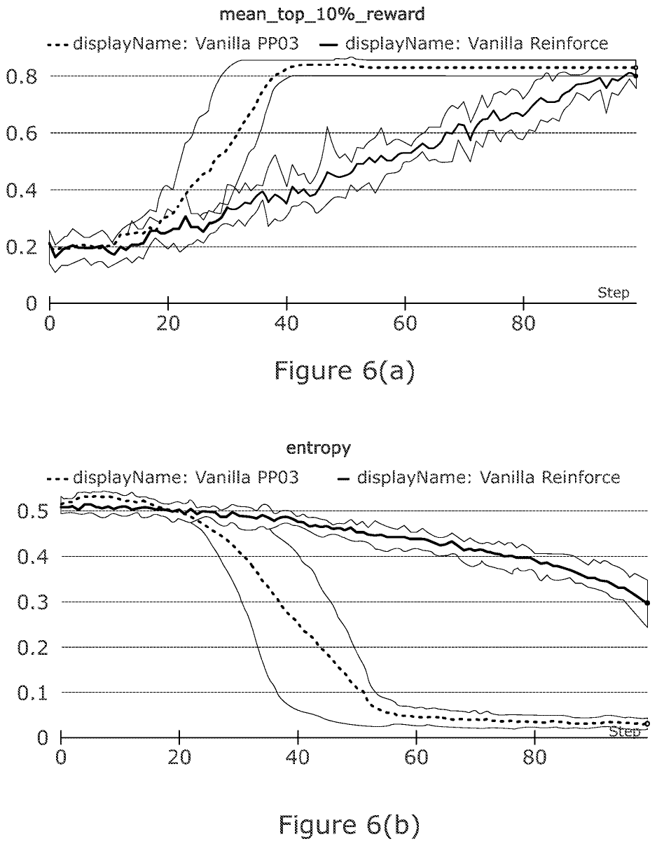

FIG. 6 illustrates an example of the performance of a proximal policy optimisation (PPO) method to optimise the generative model against a standard so-called REINFORCE algorithm (as described in “Simple statistical gradient-following algorithms for connectionist reinforcement learning”, Williams, Machine Learning 8, 229-256 (1992)). In particular, FIG. 6(a) shows mean reward signal of the top 10% of generated molecules against number of iterations, and FIG. 6(b) shows the entropy of generated molecules against number of iterations. As may be seen, PPO outperforms REINFORCE and provides faster convergence to a better/optimal policy.

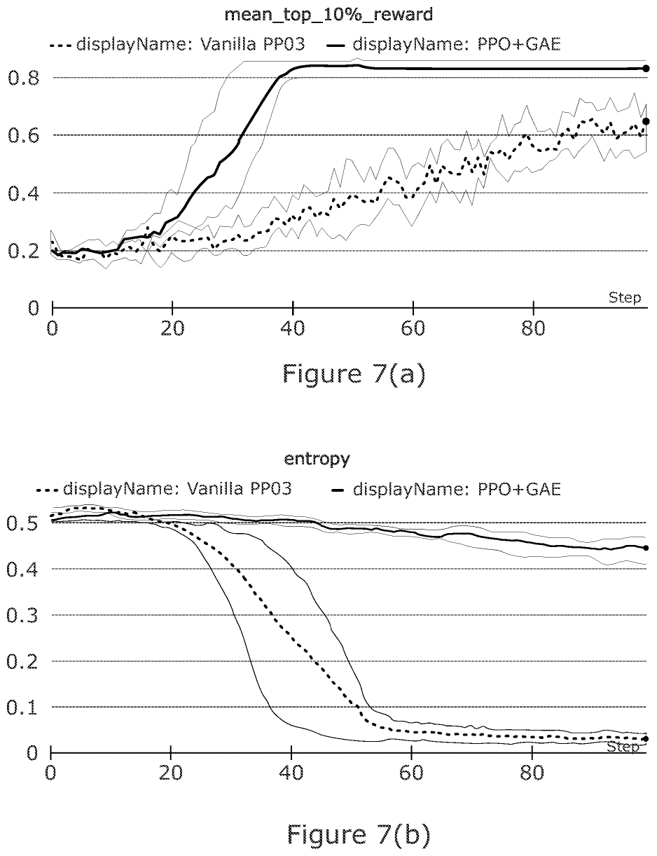

FIGS. 7(a) and 7(b) show mean reward signal of the top 10% of generated molecules and entropy, respectively, against number of iterations in an illustrative example of the performance of a standard (‘vanilla’) PPO method against PPO with generalised advantage estimation (GAE). The addition of GAE helps to reduce the variance of generated molecules and results in more stable learning curves.

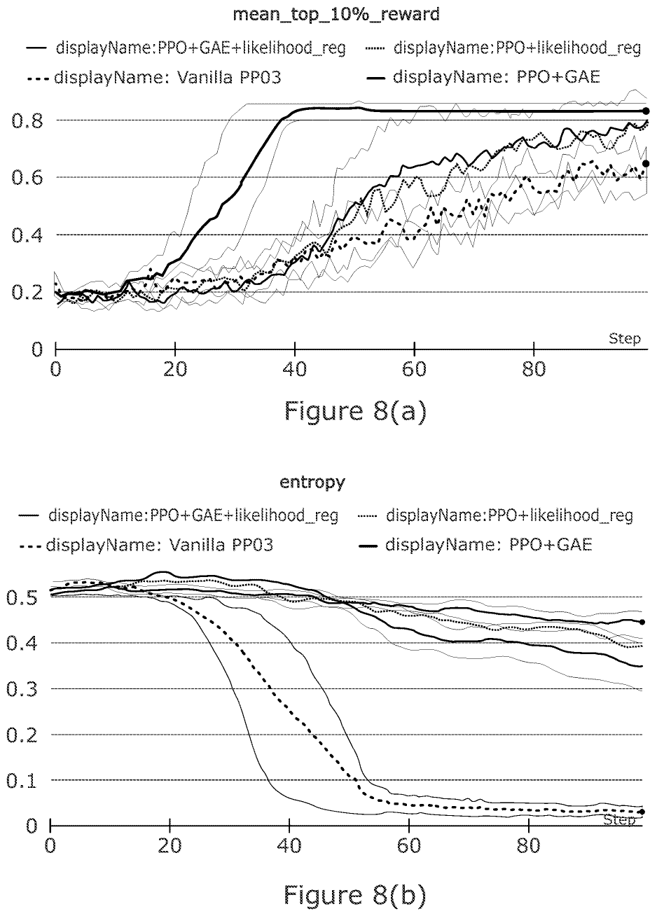

FIGS. 8(a) and 8(b) show mean reward signal of the top 10% of generated molecules and entropy, respectively, against number of iterations in an illustrative example of the performance of different RL methods. In particular, FIG. 8 compares the performance of: standard (‘vanilla’) PPO; PPO with GAE; PPO with likelihood regularisation; and, PPO with GAE and likelihood regularisation. The inclusion of likelihood regularisation (optionally with GAE) encourages the model to generate more diverse molecules.

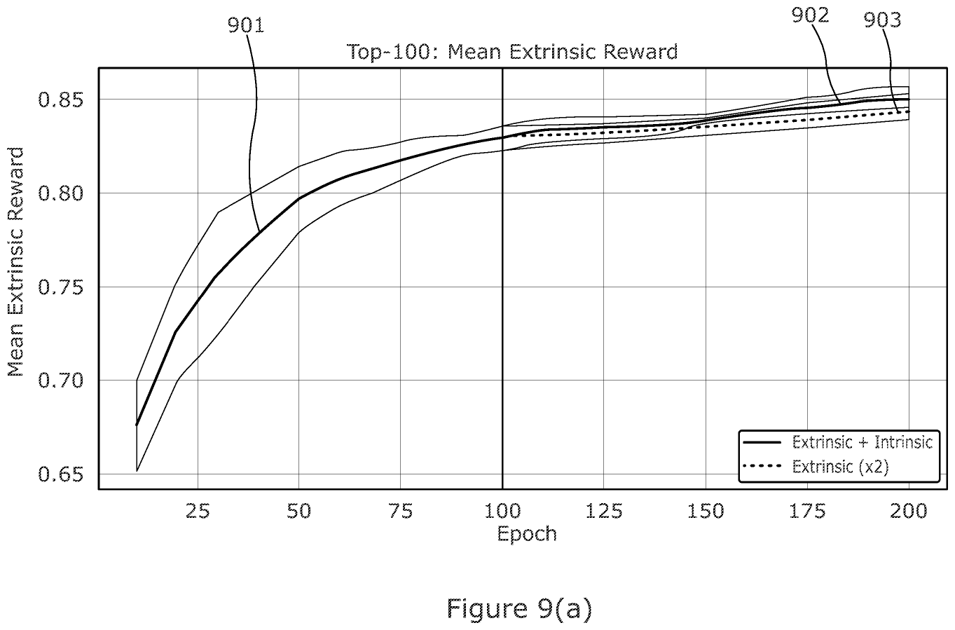

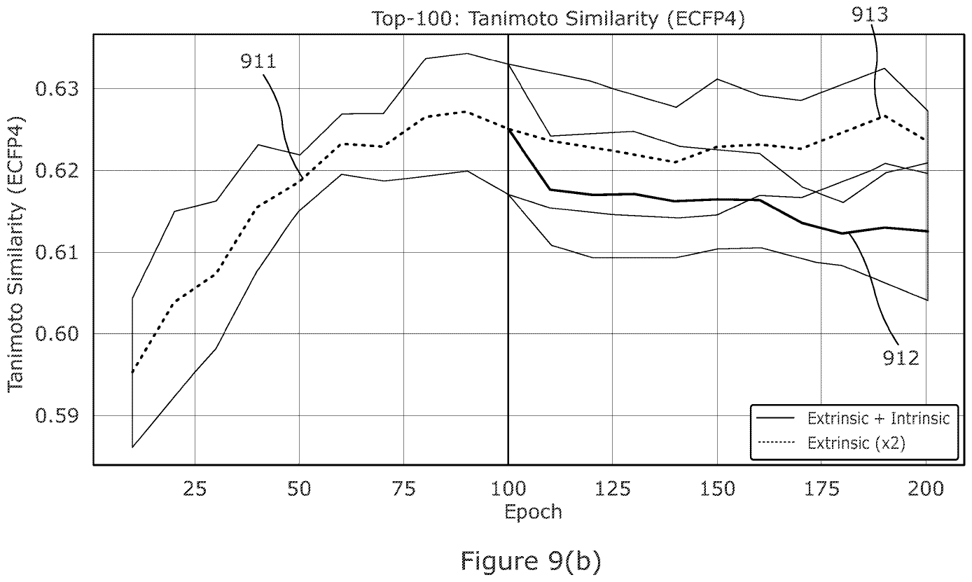

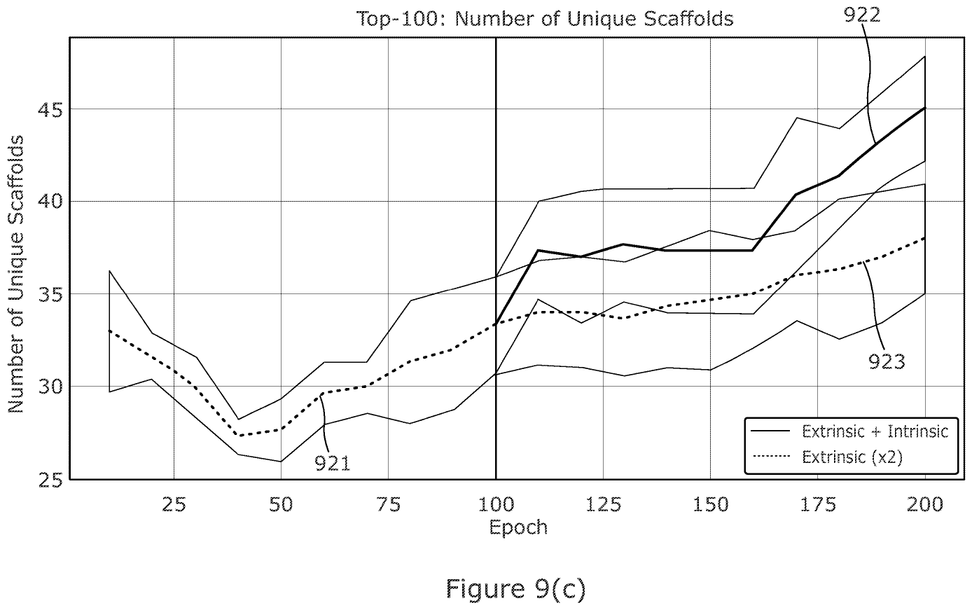

FIGS. 9(a), 9(b) and 9(c) shows mean extrinsic reward signal, average pairwise Tanimoto similarity (using ECFP4 fingerprints), and number of unique (Bemis-Murcko) scaffolds, respectively, against number of epochs in an illustrative example of the described method where a combination of extrinsic and intrinsic rewards are used to generate compounds. In this example, the compounds generated (by the generative model) in the first 100 epochs of the method were scored only using extrinsic rewards. These plots are labelled 901, 911, 921 in FIGS. 9(a)-9(c), respectively. After 100 epochs, the generated compounds were scored either: using a combination of extrinsic and intrinsic rewards, labelled as plots 902, 912, 922 in FIGS. 9(a)-9(c), respectively; or, by resetting the prior in the generative model and running again with extrinsic-only rewards with a different random seed, labelled as plots 903, 913, 923 in FIGS. 9(a)-9(c), respectively. In the described example, the top-100 scoring compounds (according to the compound score, i.e. extrinsic reward) of the compounds generated by the generative model are used to calculate the mean reward signal, Tanimoto similarity and number of unique scaffolds. The standard deviation of each plot is also shown.

It may be seen from FIG. 9(a) that the mean extrinsic reward of the top-100 scoring compounds using extrinsic and intrinsic rewards is greater than that of the top-100 scoring compounds using extrinsic-only rewards, i.e. plot 902 becomes steadily greater than plot 903 (after epoch 100). It may also be seen from FIG. 9(b) that the similarity of the top-100 scoring compounds decreases for subsequent epochs when using extrinsic and intrinsic rewards, i.e. plot 912 becomes steadily greater than plot 913 (after epoch 100). Furthermore, it is seen from FIG. 9(c) that the number of unique scaffolds of the top-100 scoring compounds using extrinsic and intrinsic rewards is greater than that of the top-100 scoring compounds using extrinsic-only rewards, i.e. plot 922 becomes steadily greater than plot 923 (after epoch 100). That is, the compounds generated when extrinsic and intrinsic rewards are used are increasingly dissimilar from one another. Specifically, the example illustrates that the described method allows for more diverse compounds/molecules to be generated (from different, unexplored parts of compound space) by the generative model while still ensuring that those generated compounds score highly against a desired property profile for generated compounds (e.g. against the property profile of a candidate compound).

In the example illustrated in FIGS. 9(a)-9(c), the extrinsic reward Rₑₓₜ of a generated compound is calculated as follows:

$${R}_{e x t} = \frac{1}{8.5} ( 3 {f}_{1} ( {\hat{x}}_{1} ) + 2 {f}_{2} ( {\hat{x}}_{2} ) + {f}_{3} ( {x}_{M W} ) + {f}_{4} ( {x}_{A R C} ) + 1.5 * {x}_{Q E D} )$$

In the above, \vec{x}₁ϵ [symbol] is potency (PIC50) against a target, as predicted by a defined Random Forest predictive model, \hat{x}₂ϵ [symbol] is an acid dissociation constant (pKa), as predicted by a defined RNN model, x_{MW} [symbol] is calculated molecular weight, x_{ARC} [symbol] is the calculated aromatic ring count, and x_{QED}ϵ[0,1] is quantitative estimate of drug-likeness as outlined above. The functions

$${\{ {f}_{i} : ℝ \to [ 0 , 1 ] \}}_{i = 1}^{4}$$

are nonlinear desirability transformations used to specify desirable ranges for each property. These transformations map to the range [0,1], where 0 is given to least desirable values and 1 is given to most desirable. In the described example, the intrinsic reward is given by the expression for \hat{r}(m) defined above. The combined reward/score is a linear combination of weighted values of Rₑₓₜ and \hat{r}(m).

Tables 1 and 2 illustrates results/metrics obtained on the outputs of different PPO methods in accordance with examples of the invention for different reward functions, where these metrics are calculated on the top 1000 output molecules and top 2500 output molecules in Table 1 and Table 2, respectively, where the output molecules have been ranked in terms of their rewards as computed by the reward function.

The ‘Avg. Score’ metric evaluates how good the molecules are according to the reward function. The ‘Avg. FPSim’ and ‘Num. Scaffolds’ metrics are both indicators of how diverse the set of molecules is. The ‘best value’ for each metric and each reward function/objective are indicated in bold.

Four different reward functions/objectives are considered, i.e. RF1, RF2, RF3, RF4. Three different methods are considered. The first column relates to the use of extrinsic rewards only, as described above. The second column also relates to the use of extrinsic rewards only, but combines the results of two separate runs of the method. The third column relates to the use of both extrinsic and intrinsic rewards, as described above. In practice, this involves running the method with extrinsic rewards only and then running the method with a linear combination of extrinsic and intrinsic rewards.

Across all of the different objectives RF1, RF2, RF3, RF4, it is seen that the extrinsic/intrinsic rewards combination approach (third column) outputs a more diverse range of scaffolds than other approaches for the top 1000 molecules. This approach therefore produces a more diverse, higher-scoring et of molecular structures.

For the first two objectives, RF1 and RF2, the extrinsic/intrinsic rewards combination approach (third column) outputs have a lower average score than the second approach of running the method with extrinsic rewards twice. However, the diversity of the third column's approach is greatly improved. In these cases, the approach of the method in the third column has been encouraged to stay away from the mode discovered in the first run of the method, but has successfully found a more diverse set of molecules with similar scores to the first run.

For the second two objectives, RF3 and RF4, the extrinsic/intrinsic rewards combination approach (third column) outputs are slightly less diverse, but have a higher average score than the other approaches. For these objectives, the third column's approach successfully discovered and explored a new, higher-scoring region of molecular space. The reduction in diversity may be explained by the method generating many similar compounds from this new mode, and a lot of the compounds from the first run (extrinsic rewards only) being dropped from the top 1000 or top 2500. This is beneficial as higher-quality compounds are obtained compared to running the method with extrinsic rewards only, e.g. as in the first column.

TABLE 1

| | Extrinsic Rewards Only | | | Extrinsic Rewards Only (×2) | | | Extrinsic + Intrinsic Rewards | | |
|---|---|---|---|---|---|---|---|---|---|
| Reward | Avg. | Avg. | Num. | Avg. | Avg. | Num. | Avg. | Avg. | Num. |
| Function | Score | FPSim | Scaffolds | Score | FPSim | Scaffolds | Score | FPSim | Scaffolds |
| RF1 | 0.974 | 0.176 | 772 | 0.977 | 0.183 | 744 | 0.974 | 0.167 | 786 |
| RF2 | 0.553 | 0.158 | 136 | 0.572 | 0.154 | 114 | 0.564 | 0.143 | 156 |
| RF3 | 0.325 | 0.323 | 543 | 0.359 | 0.324 | 573 | 0.512 | 0.338 | 638 |
| RF4 | 0.499 | 0.315 | 275 | 0.519 | 0.319 | 271 | 0.523 | 0.313 | 280 |

TABLE 2

| | Rogue | | | Rogue (×2) | | | Rogue-Explore | | |
|---|---|---|---|---|---|---|---|---|---|
| Reward | Avg. | Avg. | Num. | Avg. | Avg. | Num. | Avg. | Avg. | Num. |
| Function | Score | FPSim | Scaffolds | Score | FPSim | Scaffolds | Score | FPSim | Scaffolds |
| RF1 | 0.968 | 0.167 | 1864 | 0.973 | 0.171 | 1834 | 0.969 | 0.154 | 1935 |
| RF2 | 0.537 | 0.150 | 296 | 0.560 | 0.150 | 220 | 0.551 | 0.137 | 331 |
| RF3 | 0.270 | 0.315 | 1109 | 0.303 | 0.317 | 1170 | 0.461 | 0.332 | 1057 |
| RF4 | 0.441 | 0.306 | 658 | 0.463 | 0.307 | 658 | 0.484 | 0.304 | 578 |

One or more of the generated new compounds may then be synthesised and tested to determine one or more of their properties, such as biological, biochemical, physiological and/or pharmacological properties. One or more of the generated new compounds may be included in a population of compounds from which a trained machine learning model selects one or more of the compounds for synthesis, e.g. one or more compounds from the population predicted by the model to have certain desired properties. For instance, the selected or predicted compounds may be a candidate drug or therapeutic molecule having a desired activity against a predetermined target molecule, where the predetermined target molecule is an in vitro and/or in vivo therapeutic, diagnostic or experimental assay target. In particular, the method may be used to generate/design compounds that score highly relative to the desired property profile for a particular drug design project. In turn, this means that the number of iterations of synthesis and testing may be reduced as candidate compounds are likely to be created in fewer cycles, thereby saving on time and costs.

The method of the invention may be implemented on any suitable computing device, for instance by one or more functional units or modules implemented on one or more computer processors. Such functional units may be provided by suitable software running on any suitable computing substrate using conventional or customer processors and memory. The one or more functional units may use a common computing substrate (for example, they may run on the same server) or separate substrates, or one or both may themselves be distributed between multiple computing devices. A computer memory may store instructions for performing the method, and the processor(s) may execute the stored instructions to perform the method.

Many modifications may be made to the above-described examples without departing from the spirit and scope of the invention as defined herein with particular reference to the appended clauses and claims.

### CLAUSES

1. A method for de novo drug design, the method comprising computer-implemented steps of:
   receiving a training set of a plurality of training compounds, each training compound having a plurality of structural features;
   defining a desired property profile indicative of one or more desired properties of a drug compound to be designed;
   training a generative model using the training set to obtain a prior probability distribution of the structural features in the training set;
   generating an initial set of one or more compounds using the generative model, wherein generating each compound comprises sampling the prior probability distribution;
   determining, using a defined reward function, a compound score for each compound in the initial set, the compound score being indicative of a proximity of a property profile of the respective compound to the desired property profile;
   determining a current policy of the generative model, the current policy being determined by modifying the prior probability distribution based on the determined compound scores using a reinforcement learning algorithm; and,
   generating a new set of one or more compounds using the current policy of the generative model,
wherein the training set comprises:
   a first training set of a first plurality of training compounds; and,
   a second training set of a second plurality of training compounds, the second training set being different from the first training set, and wherein training the generative model comprises:
   using the first training set to obtain a first prior probability distribution of the structural features in the first training set; and,
   using the second training set to modify the first prior probability distribution to obtain the prior probability distribution, wherein the prior probability distribution incorporates a distribution of the structural features in the second training set.
2. A method according to Clause 1, the method comprising:
   determining the compound score for each compound in the new set;
   updating the current policy based on the determined compound scores for the compounds in the new set;
   generating a new set of one or more compounds using the updated current policy.
3. A method according to Clause 2, comprising determining the compound score for each compound in the new set generated using the updated current policy.
4. A method according to Clause 3, comprising iteratively repeating the steps of:
   updating the current policy based on the determined compound scores for the compounds in the new set generated using the current policy;
   generating a new set of one or more compounds using the updated current policy; and,
   determining the compound score for each compound in the new set generated using the updated current policy, until a stop condition is satisfied.
5. A method according to Clause 4, wherein the stop condition includes one or more of:
   a prescribed number of iterations have been performed;
   the compound score of one or more compounds in the new set satisfies a threshold condition; optionally, wherein the threshold condition is that the compound score of one or more compounds in the new set is greater than a defined threshold compound score;
   a rate of change of the compound scores across iterations falls below a prescribed level.
6. A method according to any previous clause, wherein determining the compound score comprises:
   applying a plurality of property scorer functions to the respective compound to obtain a plurality of property scores for the compound, each property score being indicative of a proximity of a value of one of the properties of the compound to a desired value of the respective property in the desired property profile;
   applying the reward function to the plurality of obtained property scores to obtain the compound score for the respective compound; optionally, wherein applying the reward function comprises obtaining a linear combination of the obtained property scores; further optionally, wherein each obtained property score is weighted prior to obtaining the linear combination.
7. A method according to any previous clause, wherein the compound score is a one-dimensional value.
8. A method according to any previous clause, wherein using the reinforcement learning algorithm comprises applying a policy gradient method to minimise a defined loss function.
9. A method according to Clause 8, wherein applying the policy gradient method comprises applying a proximal policy optimisation method, which comprises including one or more terms in the loss function defining clipped probability ratios.
10. A method according to Clause 8 or Clause 9, wherein applying the policy gradient method comprises including a Kullback-Leibler divergence term in the loss function.
11. A method according to any of Clauses 8 to 10, wherein applying the policy gradient method comprises including a likelihood regularisation term in the loss function.
12. A method according to any of Clauses 8 to 11, wherein applying the policy gradient method comprises including a maximum mean discrepancy term in the loss function.
13. A method according to Clause 12, wherein including the maximum mean discrepancy term in the loss function comprises defining a metric to measure a difference between two trajectory distributions indicative of respective compounds, wherein the compounds are represented as extended connectivity fingerprints (ECFP), and wherein the metric is defined to determine a kernel of similarity between the compounds.
14. A method according to Clause 13, wherein the kernel of similarity is defined to be a Tanimoto similarity.
15. A method according to any of Clauses 8 to 14, wherein applying the policy gradient method comprises including each of: a Kullback-Leibler divergence term; a likelihood regularisation term; and, a maximum mean discrepancy term, in the loss function.
16. A method according to any of Clauses 8 to 15, wherein applying the policy gradient method comprises applying a generalised advantage estimation scheme.
17. A method according to any previous clause, wherein the second training set comprises fewer compounds than the first training set.
18. A method according to any previous clause, wherein the second training set is composed of compounds that exhibit at least some desired properties in accordance with the desired property profile; optionally wherein the second training set is composed of compounds of a defined type; further optionally wherein the defined type is a kinase.
19. A method according to any previous clause, wherein the first training set is composed of compounds from a defined region of compound space, and the second training set is composed of compounds from a sub-region of the defined region of compound space.
20. A method according to any previous clause, wherein the generative model is a recurrent neural network model.
21. A method according to any previous clause, wherein the training compounds and generated compounds are represented as SMILES strings.
22. A method according to any of Clauses 1 to 20, wherein the generated compounds are represented as CHUCKLES strings.
23. A method according to any of Clauses 1 to 20, wherein the plurality of compounds in the received training set are each represented as SMILES strings, the method comprising converting the SMILES strings to represent the plurality of compounds as CHUCKLES strings, and wherein the generative model is trained using the plurality of compounds represented as CHUCKLES strings.
24. A method according to any of Clauses 1 to 20, wherein the generated compounds are represented as SELFIES strings.
25. A method according to any of Clauses 1 to 20, wherein the plurality of compounds in the received training set are each represented as SMILES strings, the method comprising converting the SMILES strings to represent the plurality of compounds as SELFIES strings, and wherein the generative model is trained using the plurality of compounds represented as SELFIES strings.
26. A method according to any previous clause, wherein generating each compound comprises sequentially sampling the current policy to obtain a string of tokens representing the generated compound.
27. A method according to Clause 26, wherein the current policy is sampled until a token indicating that the generation process is complete is sampled.
28. A method according to Clause 26 or Clause 27, wherein generating each compound comprises:
   obtaining a partial string of tokens representing a partial compound;
   evaluating the partial compound relative to the desired property profile;
   updating the current policy based on the evaluation of the partial compound; and,
   sequentially sampling the updated current policy starting from the partial string of tokens.
29. A method according to Clause 28, wherein a final token of the partial string is a dot, indicating that the partial compound is a valid compound in CHUCKLES notation.
30. A method according to any of Clauses 26 to 29, wherein a subset of tokens in the string of tokens is predetermined, indicating that part of the generated compound is predetermined.
31. A method according to Clause 30, wherein the part of the generated compound corresponding to its core is predetermined.
32. A method according to Clause 31, wherein the current policy comprises a core policy and an agent policy, and wherein generating each compound comprises sampling the core policy at locations of the generated string corresponding to the core of the compound and sampling the agent policy at locations of the string corresponding to non-core parts of the compound.
33. A method according to Clause 32, wherein the core policy is fixed, and wherein updating the current policy comprises updating only the agent policy.
34. A method according to Clause 33, the method comprising tracking a policy mask during the compound generation step, the policy mask being indicative at each sampling instance of whether the core policy or agent policy is sampled to obtain a given token in the string of tokens representing the generated compound, and wherein the current policy is updated based on tokens sampled in accordance with the agent policy.
35. A method according to any previous clause, wherein the one or more desired properties includes one or more of a desired: activity; selectivity; toxicity; absorption; distribution; metabolism; and, excretion.
36. A method according to any previous clause, the method comprising selecting one or more of the generated compounds for synthesis.
37. A method according to Clause 36, wherein the selection is based on the determined compound score of the generated compounds; optionally, wherein a plurality of the generated compounds having the highest compound score are selected.
38. A method according to Clause 36, the method comprising approximating one or more properties of the generated compounds as a function of their structural features using a trained machine learning model, wherein the selection is based on the approximated properties of the respective generated compounds; optionally, wherein the trained machine learning model is a Bayesian model; further optionally, wherein the Bayesian model is a Gaussian Process model.
39. A method according to any of Clauses 36 to 38, comprising synthesising the selected one or more generated compounds.
40. A method according to Clause 39 when dependent on Clause 38, comprising retraining the machine learning model based on an ML training set including the synthesised one or more generated compounds.
41. A method according to Clause 39 or Clause 40, comprising adding the synthesised one or more generated compounds to the training set to obtain an updated training set, and retraining the generative model using the updated training set; optionally, wherein the synthesised compounds are added to the second training set.
42. A method according to any of Clauses 39 to 41, comprising:
   updating the second training set by adding at least one of the synthesised compounds;
   re-training the generative model using the updated second training set to modify the first prior probability distribution to obtain an updated prior probability distribution;
   generating an updated initial set of one or more compounds using the updated generative model, wherein generating each compound comprises sampling the updated prior probability distribution;
   determining, using the defined reward function, a compound score for each compound in the updated initial set, the compound score being indicative of a proximity of a property profile of the respective compound to the desired property profile;
   determining a current policy of the updated generative model, the current policy being determined by modifying the updated prior probability distribution based on the determined compound scores using the reinforcement learning algorithm; and, generating a new set of one or more compounds using the current policy of the updated generative model.
43. A method according to Clause 42, comprising: analysing the properties of the synthesised compounds relative to the desired property profile; and, adding only those synthesised compounds to the second training set that are within a defined proximity of the desired property profile.
44. A method according to any previous clause, comprising:
   defining a top set of a defined plurality of the generated compounds with the highest compound scores; and,
   training, using the top set, a Gaussian Process model for approximating the compound score of compounds as a function of the one or more structural features of said compounds,
the method comprising, for each generated compound:
   executing the trained Gaussian Process model to obtain an uncertainty value associated with the approximated compound score, the uncertainty value being indicative of how dissimilar the respective generated compound is relative to the compounds in the top set;
   determining a further compound score using a defined further reward function, the further compound score being a defined combination of the compound score and the obtained uncertainty value,
the method further comprising:
   updating the current policy based on the determined further compound scores; and,
   generating a new set of one or more compounds using the updated current policy.
45. A method according to any of Clauses 36 to 44, wherein the one or more selected compounds is a candidate drug or therapeutic molecule having a desired biological, biochemical, physiological and/or pharmacological activity against a predetermined target molecule.
46. A method according to Clause 45, wherein the predetermined target molecule is an in vitro and/or in vivo therapeutic, diagnostic or experimental assay target.
47. A method according to Clause 45 or Clause 46, wherein the candidate drug or therapeutic molecule is for use in medicine; for example, in a method for the treatment of an animal, such as a human or non-human animal.
48. A method for de novo drug design, the method comprising computer-implemented steps of:
   receiving a trained generative model that includes a prior probability distribution of structural features of a training set of a plurality of training compounds each having a plurality of structural features;
   generating an initial set of one or more compounds using the generative model, wherein generating each compound comprises sampling the prior probability distribution;
   determining, using a defined reward function, a compound score for each compound in the initial set, the compound score being indicative of a proximity of a property profile of the generated initial compound relative to the desired property profile;
   determining a current policy of the generative model, the current policy being determined by modifying the prior probability distribution based on the determined score using a reinforcement learning algorithm; and,
   generating a new set of one or more compounds using the current policy of the generative model.
49. A method according to Clause 48, the method comprising: receiving the training set of the plurality of training compounds; and, training the generative model using the training set to obtain the prior probability distribution.
50. A method according to Clause 49, wherein training the generative model using the training set comprises modifying a predefined probability distribution based on the training set to obtain the prior probability distribution.
51. A method according to any of Clauses 48 to 50, wherein determining the current policy comprises applying a proximal policy optimisation method to minimise a defined loss function that comprises one or more terms defining clipped probability ratios, and wherein applying the proximal policy optimisation method comprises including at least one of the following in the loss function: a Kullback-Leibler divergence term; a likelihood regularisation term; and, a maximum mean discrepancy term; optionally, wherein the loss function includes each of the Kullback-Leibler divergence term, the likelihood regularisation term, and the maximum mean discrepancy term.
52. A method according to any of Clauses 48 to 51, wherein the generated compounds are represented as SELFIES strings.
53. A method according to any of Clauses 48 to 52, wherein generating each compound comprises sequentially sampling the current policy to obtain a string of tokens representing the respective compound, and wherein a subset of tokens in the string of tokens is predetermined, indicating that part of the generated compound is predetermined.
54. A computer-implemented method for de novo drug design, the method comprising iteratively performing steps of:
   generating a set of a plurality of compounds using a defined generative model, wherein generating each compound comprises sampling a current policy of the generative model, the current policy including a probability distribution of compound structural features;
   determining, using a defined reward function, a compound score for each compound in the set, the compound score being indicative of a proximity of a property profile of the respective compound to a defined property profile indicative of one or more desired properties of a drug compound to be designed;
   storing the set of generated compounds along with the associated determined compound scores; and,
   updating the current policy of the generative model by modifying the probability distribution based on the determined compound scores using a reinforcement learning algorithm,
for a predefined number of iterations,
the method comprising:
   selecting a plurality of compounds from the stored sets of generated compounds, the selected compounds being the generated compounds having the highest compound scores;
   training, using the selected plurality of compounds, a Gaussian Process model for approximating the compound score of compounds as a function of structural features of said compounds,
the method comprising:
   generating a further set of one or more compounds by sampling the current policy of the generative model;
   executing the trained Gaussian Process model for each generated compound in the further set to obtain an uncertainty value associated with a predicted output of the Gaussian Process model, the uncertainty value being indicative of dissimilarity of the respective compound relative to the selected plurality of compounds;
   determining a further compound score for each generated compound in the further set based on a combination of the compound score and the uncertainty value for the respective compound.
55. A method according to Clause 54, wherein the compound score of each generated compound in the further set is determined using the defined reward function and/or obtained from the executed Gaussian Process model.
56. A method according to Clause 54 or Clause 56, comprising: updating the current policy of the generative model by modifying the probability distribution based on the determined further compound scores using the reinforcement learning algorithm; generating a set of one or more compounds by sampling the current policy of the generative model; determining the further compound score for each generated compound in the further set; and, storing the further set of generated compounds along with the associated determined further compound scores.
57. A method according to Clause 56, comprising:
   selecting a further plurality of compounds from the stored sets and further sets of generated compounds, the selected compounds being the generated compounds having the highest compound scores or further compound scores;
   re-training, using the selected further plurality of compounds, the Gaussian Process model for approximating the compound score of compounds as a function of structural features of said compounds.
58. A compound identified by the method of any previous clause.
59. A non-transitory, computer-readable storage medium storing instructions thereon that when executed by a computer processor causes the computer processor to perform the method of any of Clauses 1 to 57.
60. A computing device for computational drug design, comprising:
   an input arranged to receive a trained generative model including a prior probability distribution of structural features of a training set of a plurality of training compounds;
   a processor arranged to: generate an initial compound in accordance with the prior probability distribution using the trained generative model; define a desired property profile indicative of one or more desired properties of a drug to be designed; obtain a score associated with the generated initial compound, the score being indicative of a property profile of the generated initial compound relative to the desired property profile; determine a current policy of the generative model, the current policy being determined by modifying the prior probability distribution based on the determined score using a reinforcement learning algorithm; and, generate a new compound in accordance with the current policy; and,
   an output arranged to output the generated new compound.
61. A computing device according to Clause 60, wherein the processor is configured to perform a method according to any of Clauses 1 to 57.

1-56. (canceled)

57. A computer-implemented method comprising:
hierarchically pre-training a generative neural network model utilizing supervised learning on a first training set followed by supervised training on a second training set, wherein the first training set is larger than the second training set and the second training set is filtered to a defined type of compound;
generating, utilizing the generative neural network model, a compound by sampling from a learned prior probability distribution of the generative neural network model;
generating a reinforcement learning agent from the generative neural network model utilizing reinforcement learning by:
generating, utilizing a scoring model, a compound score for the compound, wherein the compound score indicates a proximity of a property profile of the compound relative to a target property profile; and
generating a reinforcement learning policy of the reinforcement learning agent from the learned prior probability distribution of the generative neural network model and the compound score for the compound; and
generating, utilizing the reinforcement learning agent, an additional compound based on the reinforcement learning policy.

58. The computer-implemented method of claim 57, wherein the generative neural network model comprises at least one of a recurrent neural network or a transformer model, and further comprising generating the compound by:
generating an initialization token; and
generating, utilizing at least one of the recurrent neural network or the transformer model, a compound token sequence from the initialization token by sequentially sampling from the learned prior probability distribution.

59. The computer-implemented method of claim 57, further comprising pre-training the generative neural network model utilizing supervised learning by:
generating, utilizing the generative neural network model, predicted structures from initialised representations of training compounds of a training set; and
modifying parameters of the generative neural network model by comparing the predicted structures to the training compounds of the training set.

60. The computer-implemented method of claim 57, wherein generating the compound score utilizing the scoring model comprises generating a predicted biological property of the compound utilizing a trained machine learning model.

61. The computer-implemented method of claim 57, wherein generating the compound score comprises:
generating, utilizing a first scoring model, a first compound score;
generating, utilizing a second scoring model, a second compound score; and
combining the first compound score and the second compound score to generate the compound score.

62. The computer-implemented method of claim 61, further comprising:
generating the first compound score utilizing the first scoring model to indicate the proximity of the property profile of the compound relative to the target property profile; and
generating the second compound score utilizing the second scoring model by generating a measure of dissimilarity between the compound and a set of additional compounds.

63. The computer-implemented method of claim 57, wherein generating the reinforcement learning agent comprises:
generating an augmented likelihood distribution by modulating the learned prior probability distribution of the generative neural network model by the compound score;
generating a loss by comparing the augmented likelihood distribution and the learned prior probability distribution; and
modifying parameters of the generative neural network model utilizing the loss to generate the reinforcement learning policy.

64. The computer-implemented method of claim 57, wherein generating the additional compound comprises:
generating, utilizing the generative neural network model, a first portion of the additional compound; and
generating, utilizing the reinforcement learning agent, a second portion of the additional compound.

65. A non-transitory computer-readable medium storing instructions that, when executed by at least one processor, cause a computing device to:
generate, utilizing a generative neural network model, a compound by sampling from a learned prior probability distribution of the generative neural network model, wherein the generative neural network model is pre-trained utilizing supervised learning;
generate a reinforcement learning agent from the generative neural network model utilizing reinforcement learning by:
generating, utilizing a scoring model, a compound score for the compound, wherein the compound score indicates a proximity of a property profile of the compound relative to a target property profile; and
generating, utilizing proximal policy optimization, a reinforcement learning policy of the reinforcement learning agent from the learned prior probability distribution of the generative neural network model and the compound score for the compound; and
generate, utilizing the reinforcement learning agent, an additional compound based on the reinforcement learning policy.

66. The non-transitory computer-readable medium of claim 65, wherein the generative neural network model comprises at least one of a recurrent neural network or a transformer model and the instructions further cause the computing device to generate the compound by:
generating an initialization token; and
generating, utilizing at least one of the recurrent neural network or the transformer model, a compound token sequence from the initialization token by sequentially sampling from the learned prior probability distribution.

67. The non-transitory computer-readable medium of claim 65, wherein the instructions further cause the computing device to pre-train the generative neural network model utilizing supervised learning by:
generating, utilizing the generative neural network model, predicted structures from initialised representations of training compounds of a training set; and
modifying parameters of the generative neural network model by comparing the predicted structures to the training compounds of the training set.

68. The non-transitory computer-readable medium of claim 65, wherein the instructions further cause the computing device to:
generate a first compound score utilizing a first scoring model to indicate the proximity of the property profile of the compound relative to the target property profile;
generate a second compound score utilizing an second scoring model by generating a measure of dissimilarity between the compound and a set of additional compounds; and
combining the first compound score and the second compound score to generate the compound score.

69. The non-transitory computer-readable medium of claim 65, wherein the instructions further cause the computing device to generate the reinforcement learning agent by:
generating an augmented likelihood distribution by modulating the learned prior probability distribution of the generative neural network model by the compound score;
generating a loss by comparing the augmented likelihood distribution and the learned prior probability distribution; and
modifying parameters of the generative neural network model utilizing the loss to generate the reinforcement learning policy.

70. The non-transitory computer-readable medium of claim 65, wherein the instructions further cause the computing device to generate the additional compound by:
generating, utilizing the generative neural network model, a first portion of the additional compound; and
generating, utilizing the reinforcement learning agent, a second portion of the additional compound.

71. A system comprising:
at least one processor; and
at least one non-transitory computer-readable storage medium storing instructions that, when executed by the at least one processor, cause the system to:
generate, utilizing a generative neural network model, a compound by sampling from a learned prior probability distribution of the generative neural network model, wherein the generative neural network model is pre-trained utilizing supervised learning;
generate a reinforcement learning agent from the generative neural network model utilizing reinforcement learning by:
generating a first compound score utilizing a first scoring model to indicate a proximity of a property profile of the compound relative to a target property profile;
generating a second compound score utilizing a second scoring model by generating a measure of dissimilarity between the compound and a set of additional compounds;
combining the first compound score and the second compound score to generate a compound score; and
generating a reinforcement learning policy of the reinforcement learning agent from the learned prior probability distribution of the generative neural network model and the compound score for the compound; and
generate, utilizing the reinforcement learning agent, an additional compound based on the reinforcement learning policy.

72. The system of claim 71, wherein the generative neural network model comprises at least one of a recurrent neural network or a transformer model and the instructions cause the system to generate the compound by:
generating an initialization token; and
generating, utilizing at least one of the recurrent neural network or the transformer model, a compound token sequence from the initialization token by sequentially sampling from the learned prior probability distribution.

73. The system of claim 71, wherein the instructions cause the system to pre-train the generative neural network model utilizing supervised learning by:
generating, utilizing the generative neural network model, predicted structures from initialised representations of training compounds of a training set; and
modifying parameters of the generative neural network model by comparing the predicted structures to the training compounds of the training set.

74. The system of claim 71, wherein the instructions cause the system to hierarchically pre-train the generative neural network model utilizing supervised learning on a first training set followed by supervised training on a second training set, wherein the first training set is larger than the second training set and the second training set is filtered to a defined type of compound.

75. The system of claim 71, wherein the instructions cause the system to generate the reinforcement learning agent by:
generating an augmented likelihood distribution by modulating the learned prior probability distribution of the generative neural network model by the compound score;
generating a loss by comparing the augmented likelihood distribution and the learned prior probability distribution; and
modifying parameters of the generative neural network model utilizing the loss to generate the reinforcement learning policy.

76. The system of claim 71, wherein the instructions cause the system to generate the additional compound by:
generating, utilizing the generative neural network model, a first portion of the additional compound; and
generating, utilizing the reinforcement learning agent, a second portion of the additional compound.
